(19) United States
(12) Patent Application Publication
(10) Pub. No.: US 2012/0040987 A1
(43) Pub. Date: Feb. 16, 2012

(54) DERIVATIVES OF 6-(6-NH-SUBSTITUTED-TRIAZOLOPYRIDAZINE-SULFANYL) BENZOTHIAZOLES AND BENZIMIDAZOLES, PREPARATION THEREOF, USE THEREOF AS DRUGS, AND USE THEREOF AS MET INHIBITORS

(75) Inventors: Eric Bacque, Paris (FR); Conception Nemecek, Paris (FR); Antonio Ugolini, Paris (FR); Sylvie Wentzler, Paris (FR)

(73) Assignee: SANOFI, Paris (FR)

(21) Appl. No.: 13/147,297

(22) PCT Filed: Feb. 4, 2010
(86) PCT No.: PCT/FR2010/050179

§ 371 Date: Oct. 28, 2011

(30) Foreign Application Priority Data
Feb. 6, 2009 (FR) 0900513

(51) Int. Cl. A61K 31/5025; C07D 277/82; A61P 35/00; A61P 21/00; A61P 3/10; A61P 17/06; A61P 27/02; A61P 19/02; A61P 25/00; A61P 11/06; A61P 7/02; A61P 3/00; A61P 37/08; A61P 35/04; C07D 487/04

(52) U.S. Cl. 514/248; 544/236; 548/163; 548/164

(57) ABSTRACT

The invention relates to novel products of the formula (I) where: (II) is a single or double bond; Rb is a hydrogen or fluorine atom; Ra is a NH-Rc radical in which Rc is an optionally substituted heterocycloalkyl, aryl, heteroaryl or -alkylcycloalkyl radical; X is S, SO, or SO2; A is NH or S; W is H, alkyl, or COR with R being cycloalkyl; alkyl; alkoxy; O-phenyl; —O— (CH2)n-phenyl with n=1 to 4; or NR1R2 with R1 being H or alk and R2 is H, cycloalkyl or alkyl; or R1, R2 form a cycle together with N optionally containing O, S, N and/or NH; all of said radicals being optionally substituted; wherein said products can be in any isomer or salt form, and can be used as drugs, in particular as MET inhibitors.

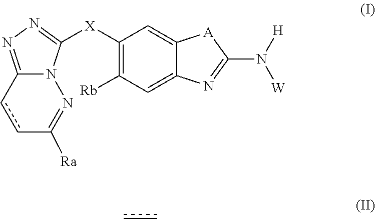

The present invention relates to novel 6-(6-NH-substituted triazolopyridazine sulphanyl)benzothiazole and 6-(6-NH-substituted triazolopyridazine sulphanyl)benzimidazole derivatives, to a process for preparing them, to the novel intermediates obtained, to their use as medicaments, to pharmaceutical compositions containing them and to the novel use of such 6-(6-NH-substituted triazolopyridazine sulphanyl)benzothiazole and 6-(6-NH-substituted triazolopyridazine sulphanyl)benzimidazole derivatives.

The present invention relates more particularly to novel 6-(6-NH-substituted triazolopyridazine sulphanyl)benzothiazole and 6-(6-NH-substituted triazolopyridazine sulphanyl)benzimidazole derivatives having an anticancer activity, via the modulation of the activity of proteins, in particular kinases.

To date, most of the commercial compounds used in chemotherapy are cytotoxic, which poses major problems of side effects and of patient tolerance. These effects could be limited if the medicaments used act selectively on cancer cells, to the exclusion of healthy cells. One of the solutions for limiting the adverse effects of a chemotherapy may thus consist in using medicaments that act on metabolic pathways or constituent elements of these pathways, predominantly expressed in the cancer cells, and which would be sparingly expressed or not expressed in healthy cells. The protein kinases are a family of enzymes that catalyze the phosphorylation of hydroxyl groups of specific residues of proteins, such as tyrosine, serine or threonine. Such phosphorylations can largely modify the function of proteins: thus, protein kinases play an important role in regulating a wide variety of cell processes, including in particular metabolism, cell proliferation, cell adhesion and motility, cell differentiation or cell survival, certain protein kinases playing a central role in the initiation, development and accomplishment of cell cycle events.

Among the various cellular functions in which the activity of a protein kinase is involved, certain processes represent attractive targets for treating certain diseases. As an example, mention may in particular be made of angiogenesis and the control of the cell cycle and also that of cell proliferation, in which protein kinases can play an essential role. These processes are in particular essential for the growth of solid tumours and also for other diseases: in particular molecules that inhibit such kinases are capable of limiting undesired cell proliferations such as those observed in cancers, and may play a part in preventing, regulating or treating neurodegenerative diseases such as Alzheimer's disease or neuronal apoptosis.

A subject of the present invention is novel derivatives with inhibitory effects on protein kinases. The products according to the present invention may thus in particular be used for preventing or treating diseases that may be modulated by inhibition of protein kinases.

The products according to the present invention in particular show anticancer activity, via the modulation of the activity of kinases. Among the kinases for which a modulation of the activity is sought, MET and also mutants of the MET protein are preferred.

The present invention also relates to the use of said derivatives for the preparation of a medicament for use in human therapy.

Thus, one of the objects of the present invention is to provide compounds that have an anticancer activity, by acting in particular on kinases. Among the kinases for which a modulation of the activity is sought, MET is preferred.

In the pharmacological section hereinafter, it is shown, in biochemical tests and on cell lines, that the products of the present invention thus inhibit in particular the autophosphorylation activity of MET and the proliferation of cells whose growth depends on MET or on mutant forms thereof.

MET, or Hepatocyte Growth Factor Receptor, is a receptor with tyrosine kinase activity, expressed in particular by epithelial and endothelial cells. HGF, Hepatocyte Growth Factor, is described as the specific ligand for MET. HGF is secreted by mesenchymal cells and activates the MET receptor, which homodimerizes. Consequently, the receptor autophosphorylates on the tyrosines of the catalytic region Y1230, Y1234 and Y1235.

Stimulation of MET with HGF induces cell proliferation, scattering (or dispersion) and motility, resistance to apoptosis, invasion and angiogenesis.

MET and likewise HGF are found to be overexpressed in many human tumours and a wide variety of cancers. MET is also found to be amplified in gastric tumours and glyoblastomas. Many point mutations of the MET gene have also been described in tumours, in particular in the kinase domain, but also in the juxtamembrane domain and the SEMA domain. Overexpression, amplification or mutations cause constitutive activation of the receptor and deregulation of its functions.

The present invention thus relates in particular to novel inhibitors of the MET protein kinase and of its mutants, that can be used for antiproliferative and antimetastatic treatment, in particular in oncology.

The present invention also relates to novel inhibitors of the MET protein kinase and of its mutants, that can be used for an anti-angiogenic treatment, in particular in oncology.

A subject of the present invention is the products of formula (I):

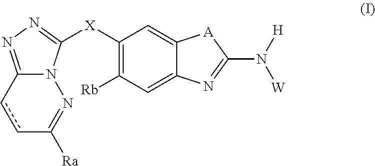

in which [symbol]
represents a single or double bond;
Rb represents a hydrogen atom or a fluorine atom;
Ra represents an —NH-Rc radical in which Rc represents an optionally substituted heterocycloalkyl, aryl, heteroaryl or alkylcycloalkyl radical;
X represents S, SO or SO₂;
A represents NH or S;
W represents a hydrogen atom; an alkyl or cycloalkyl radical optionally substituted with alkoxy, heterocycloalkyl or NR3R4; or the COR radical in which R represents:
a cycloalkyl radical, or an alkyl radical, optionally substituted with an NR3R4 radical or with an alkoxy, hydroxyl, phenyl, heteroaryl or heterocycloalkyl radical, themselves optionally substituted;
an alkoxy radical optionally substituted with NR3R4, alkoxy, hydroxyl or with heterocycloalkyl; an O-phenyl radical or an O—(CH₂)ₙ-phenyl radical, with phenyl being optionally substituted and n representing an integer from 1 to 4;
or the NR1R2 radical in which R1 and R2 are such that one of R1 and R2 represents a hydrogen atom, a cycloalkyl radical or an alkyl radical and the other of R1 and R2 represents a hydrogen atom, a cycloalkyl radical or an alkyl radical optionally substituted with one or more radicals, which may be identical or different, chosen from hydroxyl, alkoxy, heteroaryl, heterocycloalkyl, NR3R4 and optionally substituted phenyl radicals, or else R1 and R2 form, with the nitrogen atom to which they are attached, a cyclic radical containing from 3 to 10 members and optionally one or more other heteroatoms chosen from O, S, N and NH, this radical, including the possible NH that it contains, being optionally substituted;
   with R3 and R4, which may be identical or different, representing a hydrogen atom or an alkyl radical, a cycloalkyl radical, a heteroaryl radical or a phenyl radical that are all optionally substituted, or else R3 and R4 form, with the nitrogen atom to which they are attached, a cyclic radical containing from 3 to 10 members and optionally one or more other heteroatoms chosen from O, S, N and NH, this radical, including the possible NH that it contains, being optionally substituted;
   all the alkyl, cycloalkyl, heterocycloalkyl, heteroaryl, aryl and phenyl radicals defined above and also the cyclic radicals that R1 and R2 or R3 and R4 can form with the nitrogen atom to which they are attached, being optionally substituted with one or more radicals chosen from halogen atoms, hydroxyl, oxo, alkoxy, —O—CO—R5, —COOH, COOR5, —CONH₂, CONHR5, NH₂, NHR5, NR5R5′ and —NH—CO—R5 radicals and alkyl, cycloalkyl, heterocycloalkyl, CH₂-heterocycloalkyl, phenyl, CH₂-phenyl, CO-phenyl, heteroaryl and S-heteroaryl radicals, such that, in the latter radicals, the alkyl, cycloalkyl, heterocycloalkyl, phenyl and heteroaryl radicals are themselves optionally substituted with one or more radicals chosen from halogen atoms and the radicals: hydroxyl, oxo, alkyl and alkoxy containing from 1 to 4 carbon atoms, NH₂, NHalk and N(alk)₂, all the cycloalkyl, heterocycloalkyl, heteroaryl and phenyl radicals defined above also being optionally substituted with an Si(alk)₃ radical;
   R5 and R5′, which may be identical or different, represent an alkyl or cycloalkyl radical containing at most 6 carbon atoms;
   alk represents an alkyl radical containing at most 4 carbon atoms;
   said products of formula (I) being in any of the possible racemic, enantiomeric and diastereoisomeric isomer forms, and also the addition salts with mineral and organic acids or with mineral and organic bases of said products of formula (I).

The subject of the present invention is the products of formula (I) as defined above or hereinafter in which

[symbol]

represents a single or double bond;
Rb represents a hydrogen atom or a fluorine atom;
Ra represents an —NH-Rc radical in which Rc represents an optionally substituted heterocycloalkyl radical;
X represents S, SO or SO₂,
A represents NH or S;
W represents a hydrogen atom; an alkyl radical optionally substituted with alkoxy, heterocycloalkyl or NR3R4; or the COR radical in which R represents:
a cycloalkyl radical, or an alkyl radical, optionally substituted with an NR3R4, alkoxy, hydroxyl, phenyl, heteroaryl or heterocycloalkyl radical, themselves optionally substituted;
an alkoxy radical optionally substituted with NR3R4, alkoxy, hydroxyl or with heterocycloalkyl; an O-phenyl radical or an O—(CH₂)ₙ-phenyl radical, with phenyl being optionally substituted and n representing an integer from 1 to 4;
or the NR1R2 radical in which R1 and R2 are such that one of R1 and R2 represents a hydrogen atom, a cycloalkyl radical or an alkyl radical and the other of R1 and R2 represents a hydrogen atom, a cycloalkyl radical or an alkyl radical optionally substituted with one or more radicals, which may be identical or different, chosen from hydroxyl, alkoxy, heteroaryl, heterocycloalkyl, NR3R4 and optionally substituted phenyl radicals, or else R1 and R2 form, with the nitrogen atom to which they are attached, a cyclic radical containing from 3 to 10 members and optionally one or more other heteroatoms chosen from O, S, N and NH, this radical, including the possible NH that it contains, being optionally substituted;
   with R3 and R4, which may be identical or different, representing a hydrogen atom or an alkyl radical, a cycloalkyl radical, a heteroaryl radical or a phenyl radical that is optionally substituted, or else R3 and R4 form, with the nitrogen atom to which they are attached, a cyclic radical containing from 3 to 10 members and optionally one or more other heteroatoms chosen from O, S, N and NH, this radical, including the possible NH that it contains, being optionally substituted;
   all the cycloalkyl, heterocycloalkyl, heteroaryl, aryl and phenyl radicals defined above and also the cyclic radicals that R1 and R2 or R3 and R4 can form with the nitrogen atom to which they are attached, being optionally substituted with one or more radicals chosen from halogen atoms, hydroxyl, oxo, alkoxy, —O—CO—R5, NH₂, NHalk and N(alk)₂ radicals and alkyl, cycloalkyl, heterocycloalkyl, CH₂-heterocycloalkyl, phenyl, CH₂-phenyl, CO-phenyl, heteroaryl and S-heteroaryl radicals, such that, in the latter radicals, the alkyl, cycloalkyl, heterocycloalkyl, phenyl and heteroaryl radicals are themselves optionally substituted with one or more radicals chosen from halogen atoms and the radicals: hydroxyl, oxo, alkyl and alkoxy containing from 1 to 4 carbon atoms, NH₂, NHalk and N(alk)₂,
   R5 represents an alkyl or cycloalkyl radical containing at most 6 carbon atoms;
   said products of formula (I) being in any of the possible racemic, enantiomeric and diastereoisomeric isomer forms, and also the addition salts with mineral and organic acids or with mineral and organic bases of said products of formula (I).

A subject of the present invention is the products of formula (I) as defined above or hereinafter in which [symbol] , Ra, Rb and X have the values defined in any one of the other claims and:

A represents NH or S;
W represents a hydrogen atom; an alkyl radical optionally substituted with alkoxy, heterocycloalkyl or NR3R4; or the COR radical in which R represents:
a cycloalkyl radical or an alkyl radical optionally substituted with an NR3R4, alkoxy, hydroxyl, phenyl or heterocycloalkyl radical, themselves optionally substituted;
an alkoxy radical optionally substituted with NR3R4, alkoxy, hydroxyl or with heterocycloalkyl; an O-phenyl radical or an O—(CH₂)ₙ-phenyl radical, with phenyl being optionally substituted and n representing an integer from 1 to 4;
or the NR1R2 radical, in which R1 and R2 are such that one of R1 and R2 represents a hydrogen atom or an alkyl radical and the other of R1 and R2 represents a hydrogen atom, a cycloalkyl radical or an alkyl radical optionally substituted with an alkoxy, heterocycloalkyl or NR3R4 radical; or else R1 and R2 form, with the nitrogen atom to which they are attached, a cyclic radical containing from 3 to 10 members and optionally one or more other heteroatoms chosen from O, S, N and NH, this radical, including the possible NH that it contains, being optionally substituted;
   with NR3R4 such that R3 and R4, which may be identical or different, represent a hydrogen atom or an alkyl radical, or else R3 and R4 form, with the nitrogen atom to which they are attached, a cyclic radical containing from 3 to 10 members and optionally one or more other heteroatoms chosen from O, S, N and NH, this radical, including the possible NH that it contains, being optionally substituted;
   all the cycloalkyl, heterocycloalkyl and phenyl radicals and also the cyclic radicals that R1 and R2 or R3 and R4 can form with the nitrogen atom to which they are attached, defined above, being optionally substituted with one or more radicals chosen from halogen atoms, hydroxyl, alkoxy, NH₂, NHalk and N(alk)₂ radicals and alkyl, heterocycloalkyl, CH₂-heterocycloalkyl, phenyl, CH₂-phenyl and heteroaryl radicals, such that, in the latter radicals, the alkyl, heterocycloalkyl, phenyl and heteroaryl radicals are themselves optionally substituted with one or more radicals chosen from halogen atoms and the radicals: hydroxyl, alkyl and alkoxy containing from 1 to 4 carbon atoms, NH₂, NHalk and N(alk)₂;
   said products of formula (I) being in any of the possible racemic, enantiomeric and diastereoisomeric isomer forms, and also the addition salts with mineral and organic acids or with mineral and organic bases of said products of formula (I).

A subject of the present invention is the products of formula (I) as defined above or hereinafter in which [symbol] , Ra, Rb and X have the values defined in any one of the other claims and:

A represents NH or S;
W represents a hydrogen atom; an alkyl radical optionally substituted with a heterocycloalkyl or NR3R4 radical; or the COR radical in which R represents:
a cycloalkyl radical or an alkyl radical which are optionally substituted with an NR3R4 or alkoxy radical;
an O-phenyl radical or an O—(CH₂)ₙ-phenyl radical, with phenyl being optionally substituted and n representing an integer from 1 to 2;
or the NR1R2 radical, in which R1 and R2 are such that one of R1 and R2 represents a hydrogen atom, a cycloalkyl radical or an alkyl radical and the other of R1 and R2 represents a hydrogen atom, an alkyl radical optionally substituted with a heterocyclic or NR3R4 radical, or else R1 and R2 form with the nitrogen atom to which they are attached, a cyclic radical optionally containing one or more other heteroatoms chosen from O, S, N and NH, this radical, including the possible NH that it contains, being optionally substituted;
   with NR3R4 such that R3 and R4, which may be identical or different, represent a hydrogen atom or an alkyl radical, or else R3 and R4 form, with the nitrogen atom to which they are attached, a cyclic radical optionally containing one or more other heteroatoms chosen from O, S, N and NH, this radical, including the possible NH that it contains, being optionally substituted;
   all the cycloalkyl, heterocyclic and phenyl radicals and also the cyclic radicals that R1 and R2 or R3 and R4 can form with the nitrogen atom to which they are attached, defined above, being optionally substituted with one or more radicals chosen from halogen atoms, hydroxyl, alkoxy, NH₂. NHalk and N(alk)₂ radicals and alkyl and phenyl radicals, the latter radicals themselves being optionally substituted with one or more radicals chosen from halogen atoms and the radicals: hydroxyl, alkyl and alkoxy containing from 1 to 4 carbon atoms, NH₂, NHalk and N(alk)₂;
   said products of formula (I) being in any of the possible racemic, enantiomeric and diastereoisomeric isomer forms, and also the addition salts with mineral and organic acids or with mineral and organic bases of said products of formula (I).

A subject of the present invention is the products of formula (I) as defined above or hereinafter in which A represents NH, the substituents [symbol] , Ra, Rb, X and W being chosen from all the values defined for these radicals in any one of the other claims, said products of formula (I) being in any of the possible racemic, enantiomeric and diastereoisomeric isomer forms, and also the addition salts with mineral and organic acids or with mineral and organic bases of said products of formula (O).

A subject of the present invention is the products of formula (I) as defined above or hereinafter in which A represents S, the substituents [symbol] , Ra, Rb X and W being chosen from all the values defined for these radicals in any one of the other claims, said products of formula (I) being in any of the possible racemic, enantiomeric and diastereoisomeric isomer forms, and also the addition salts with mineral and organic acids or with mineral and organic bases of said products of formula (I).

A subject of the present invention is the products of formula (I) as defined above or hereinafter corresponding to formula (Ia) or (Ib):

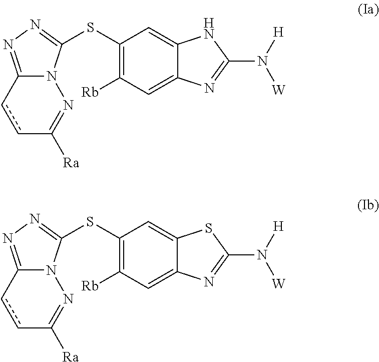

in which [symbol] , Ra, Rb and W are chosen from the meanings indicated in any one of the other claims,
said products of formula (Ia) and (Ib) being in any of the possible racemic, enantiomeric and diastereoisomeric isomer forms, and also the addition salts with mineral and organic acids or with mineral and organic bases of said products of formulae (Ia) and (Ib).

A subject of the present invention is the products of formula (I) as defined above or hereinafter in which [symbol] represents a single bond, corresponding to the products of formula (I′):

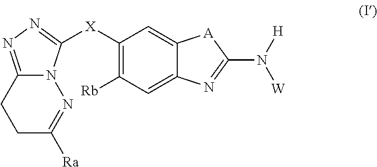

the substituents Ra, Rb, X, A and W having any one of the meanings indicated above or hereinafter,
said products of formula (I′) being in any of the possible racemic, enantiomeric and diastereoisomeric isomer forms, and also the addition salts with mineral and organic acids or with mineral and organic bases of said products of formula (I′).

A subject of the present invention is products of formula (I) as defined above or hereinafter in which [symbol] represents a double bond, corresponding to the products of formula (I″):

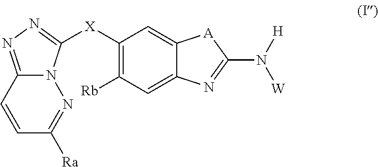

in which the substituents Ra, Rb X, A and W have any one of the meanings indicated above or hereinafter,
said products of formula (I) being in any of the possible racemic, enantiomeric and diastereoisomeric isomer forms, and also the addition salts with mineral and organic acids or with mineral and organic bases of said products of formula (I).

A subject of the present invention is the products of formula (I) as defined above or hereinafter in which [symbol] represents a single bond, corresponding to the products of formula (Ia′):

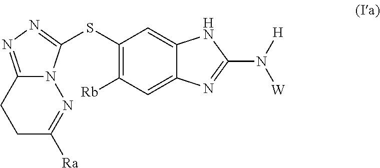

in which Ra, Rb and W are chosen from any one of the meanings indicated above or hereinafter,
said products of formula (I′a) being in any of the possible racemic, enantiomeric and diastereoisomeric isomer forms, and also the addition salts with mineral and organic acids or with mineral and organic bases of said products of formula (I′a).

A subject of the present invention is the products of formula (I) as defined above or hereinafter in which [symbol] represents a double bond, corresponding to the products of formula (I″a):

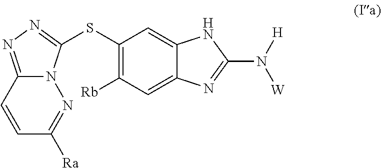

in which Ra, Rb and W are chosen from any one of the meanings indicated above or hereinafter,
said products of formula (I″a) being in any of the possible racemic, enantiomeric and diastereoisomeric isomer forms, and also the addition salts with mineral and organic acids or with mineral and organic bases of said products of formula (I″a).

A subject of the present invention is products of formula (I) as defined above or hereinafter in which [symbol] represents a single bond, corresponding to the products of formula (I′b):

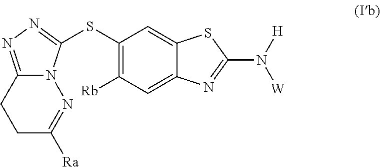

in which Ra, Rb and W are chosen from any one of the meanings indicated above or hereinafter,
said products of formula (I′b) being in any of the possible racemic, enantiomeric and diastereoisomeric isomer forms, and also the addition salts with mineral and organic acids or with mineral and organic bases of said products of formula (I′b).

A subject of the present invention is the products of formula (I) as defined above or hereinafter in which [symbol] represents a double bond, corresponding to the products of formula (I″b):

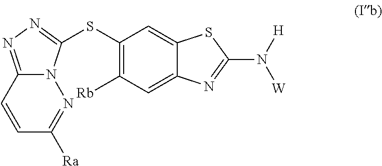

in which Ra, Rb and W are chosen from all the meanings indicated above or hereinafter,
said products of formula (I″b) being in any of the possible racemic, enantiomeric and diastereoisomeric isomer forms, and also the addition salts with mineral and organic acids or with mineral and organic bases of said products of formula (I″b).

In the products of formula (I) and in the text hereinbelow:
the term “alkyl (or Alk) radical” denotes linear and, where appropriate, branched methyl, ethyl, propyl, isopropyl, butyl, isobutyl, sec-butyl, tert-butyl, pentyl, isopentyl, hexyl, isohexyl and also heptyl, octyl, nonyl and decyl radicals and also the linear or branched positional isomers thereof: alkyl radicals containing from 1 to 6 carbon atoms and more particularly alkyl radicals containing from 1 to 4 carbon atoms of the above list are preferred;
the term “alkoxy radical” denotes linear and, where appropriate, branched methoxy, ethoxy, propoxy, isopropoxy, linear, secondary or tertiary butoxy, pentoxy or hexoxy radicals and also the linear or branched positional isomers thereof: alkoxy radicals containing from 1 to 4 carbon atoms of the above list are preferred;
the term “halogen atom” denotes chlorine, bromine, iodine or fluorine atoms, and preferably the chlorine, bromine or fluorine atom;
the term “cycloalkyl radical” denotes a saturated carbocyclic radical containing 3 to 10 carbon atoms and thus denotes in particular cyclopropyl, cyclobutyl, cyclopentyl and cyclohexyl radicals, and most particularly cyclopropyl, cyclopentyl and cyclohexyl radicals;
the term “alkylcycloalkyl radical” denotes linear and, where appropriate, branched alkyl radicals substituted with cycloalkyl radicals as defined above;
the term “heterocycloalkyl radical” thus denotes a monocyclic or bicyclic carbocyclic radical containing from 3 to 10 members, interrupted with one or more heteroatoms, which may be identical or different, chosen from oxygen, nitrogen or sulphur atoms; mention may, for example, be made of morpholinyl, thiomorpholinyl, aziridyl, azetidyl, piperazinyl, piperidyl, homopiperazinyl, pyrrolidinyl, imidazolidinyl, pyrazolidinyl, tetrahydrofuranyl, tetrahydrothienyl, tetrahydropyranyl, tetrahydrothiopyranyl, oxodihydropyridazinyl or else oxetanyl or thietanyl radicals, all these radicals being optionally substituted;
the terms “aryl” and “heteroaryl” denote monocyclic or bicyclic, unsaturated or partially unsaturated, respectively carbocyclic and heterocyclic radicals containing at most 12 members, which may optionally contain a —C(O) member, the heterocyclic radicals containing one or more heteroatoms, which may be identical or different, chosen from O, N or S with N, where appropriate, optionally substituted;
the term “aryl radical” thus denotes monocyclic or bicyclic radicals containing 6 to 12 members, such as, for example, phenyl, naphthyl, biphenyl, indenyl, fluorenyl and anthracenyl radicals, more particularly phenyl and naphthyl radicals, and even more particularly the phenyl radical. It may be noted that a carbocyclic radical containing a —C(O) member is, for example, the tetralone radical;
the term “heteroaryl radical” thus denotes monocyclic or bicyclic radicals containing 5 to 12 members: monocyclic heteroaryl radicals, for instance the radicals: thienyl such as 2-thienyl and 3-thienyl, furyl such as 2-furyl or 3-furyl, pyrannyl, pyrrolyl, pyrrolinyl, pyrazolinyl, imidazolyl, pyrazolyl, pyridyl such as 2-pyridyl, 3-pyridyl and 4-pyridyl, pyrazinyl, pyrimidinyl, pyridazinyl, oxazolyl, thiazolyl, isothiazolyl, diazolyl, thiadiazolyl, thiatriazolyl, oxadiazolyl, isoxazolyl such as 3- or 4-isoxazolyl, furazanyl or tetrazolyl, which may be free or salified, all these radicals being optionally substituted, among which more particularly the radicals: thienyl such as 2-thienyl and 3-thienyl, furyl such as 2-furyl, pyrrolyl, pyrrolinyl, pyrazolinyl, imidazolyl, pyrazolyl, oxazolyl, isoxazolyl, pyridyl, pyridazinyl, these radicals being optionally substituted; bicyclic heteroaryl radicals, for instance the radicals: benzothienyl such as 3-benzothienyl, benzothiazolyl, quinolyl, isoquinolyl, dihydroquinolyl, quinolone, tetralone, adamentyl, benzofuryl, isobenzofuryl, dihydrobenzofuran, ethylenedioxyphenyl, thianthrenyl, benzopyrrolyl, benzimidazolyl, benzoxazolyl, thionaphthyl, indolyl, azaindolyl, indazolyl, purinyl, thienopyrazolyl, tetrahydroindazolyl, tetrahydrocyclopentapyrazolyl, dihydrofuropyrazolyl, tetrahydropyrrolopyrazolyl, oxotetrahydropyrrolo-pyrazolyl, tetrahydropyranopyrazolyl, tetrahydropyridinopyrazolyl or oxodihydropyridinopyrazolyl, all these radicals being optionally substituted.

As examples of heteroaryl or bicyclic radicals, mention may more particularly be made of pyrimidinyl, pyridyl, pyrrolyl, azaindolyl, indazolyl or pyrazolyl radicals, optionally substituted with one or more substituents, which may be identical or different, as indicated above.

The carboxyl radical(s) of the products of formula (I) may be salified or esterified with the various groups known to those skilled in the art, among which mention may, for example, be made of:
among the salification compounds, mineral bases such as, for example, an equivalent of sodium, of potassium, of lithium, of calcium, of magnesium or of ammonium or organic bases such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine or N-methylglucamine,
among the esterification compounds, alkyl radicals for forming alkoxycarbonyl groups, such as, for example, methoxycarbonyl, ethoxycarbonyl, tert-butoxycarbonyl or benzyloxycarbonyl, these alkyl radicals possibly being substituted with radicals chosen, for example, from halogen atoms, and hydroxyl, alkoxy, acyl, acyloxy, alkylthio, amino or aryl radicals, such as for instance in chloromethyl, hydroxypropyl, methoxymethyl, propionyloxymethyl, methylthiomethyl, dimethylaminoethyl, benzyl or phenethyl groups.

The addition salts with mineral or organic acids of the products of formula (I) may, for example, be the salts formed with hydrochloric acid, hydrobromic acid, hydroiodic acid, nitric acid, sulphuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulphonic acids such as, for example, methanesulphonic acid, ethanesulphonic acid or propanesulphonic acid, alkyldisulphonic acids such as, for example, methanedisulphonic acid or alpha, beta-ethanedisulphonic acid, arylmonosuiphonic acids such as benzenesulphonic acid and aryldisulphonic acids.

It may be recalled that stereoisomerism can be defined in its broad sense as the isomerism of compounds having the same structural formulae, but the various groups of which are arranged differently in space, such as in particular in monosubstituted cyclohexanes, the substituent of which can be in the axial or equatorial position, and the various possible rotational conformations of ethane derivatives. However, another type of stereoisomerism exists, due to the different spatial arrangements of substituents attached either on double bonds or on rings, which is commonly known as geometrical isomerism or cis-trans isomerism. The term stereoisomers is used in the present application in its broadest sense and therefore relates to all the compounds indicated above.

The cyclic radicals that, on the one hand, R1 and R2 can form with the nitrogen atom to which they are attached and, on the other hand, R3 and R4 can form with the nitrogen atom to which they are attached are optionally substituted with one or more radicals chosen from those indicated above for the possible substituents of the heterocycloalkyl radicals, i.e. one or more radicals chosen from halogen atoms, hydroxyl, oxo, alkoxy, NH₂; NHalk and N(alk)₂ radicals, and alkyl, heterocycloalkyl, CH₂-heterocycloalkyl, phenyl, CH₂-phenyl, heteroaryl and CO-phenyl radicals, such that, in these latter radicals, the alkyl, heterocycloalkyl and phenyl radicals are themselves optionally substituted with one or more radicals chosen from halogen atoms and the radicals: hydroxyl, oxo, alkyl and alkoxy containing from 1 to 4 carbon atoms, NH₂; NHalk and N(alk)₂.

The cyclic radicals that, on the one hand, R1 and R2 can form with the nitrogen atom to which they are attached and, on the other hand, that R3 and R4 can form with the nitrogen atom to which they are attached, are in particular optionally substituted with one or more radicals, which may be identical or different, chosen from halogen atoms and alkyl, hydroxyl, alkoxy, CH₂-pyrrolidinyl, CH₂-phenyl, heteroaryl and phenyl radicals, in which the alkyl, pyrrolidinyl and phenyl radicals are themselves optionally substituted with one or more radicals, which may be identical or different, chosen from halogen atoms and alkyl, hydroxyl, oxo and alkoxy radicals.

The heterocycloalkyl radicals as defined above represent in particular azepanyl, morpholinyl, pyrrolidinyl, piperidyl, and piperazinyl radicals, themselves optionally substituted, as defined above or hereinafter.

When NR1R2 or NR3R4 forms a ring as defined above, such an amino ring may be chosen in particular from pyrrolidinyl, pyrazolidinyl, pyrazolinyl, piperidyl, azepinyl, morpholinyl or piperazinyl radicals, these radicals being themselves optionally substituted as indicated above or hereinafter: for example, with one or more radicals, which may be identical or different, chosen from halogen atoms and alkyl, hydroxyl, alkoxy, phenyl and CH₂-phenyl radicals, the alkyl or phenyl radicals being themselves optionally substituted with one or more radicals, which may be identical or different, chosen from halogen atoms and alkyl, hydroxyl and alkoxy radicals.

The NR1R2 or NR3R4 ring may more particularly be chosen from the radicals pyrrolidinyl, morpholinyl optionally substituted with one or two alkyl radicals or piperazinyl optionally substituted on the second nitrogen atom with an alkyl, phenyl and/or CH₂-phenyl radical, themselves optionally substituted with one or more radicals, which may be identical or different, chosen from halogen atoms and alkyl, hydroxyl and alkoxy radicals.

A subject of the present invention is in particular the products of formula (I) as defined above or hereinafter in which Rb represents a fluorine atom, the other substituents of the said products of formula (I) having any one of the definitions indicated above or hereinafter.

A subject of the present invention is the products of formula (I) as defined above or hereinafter in which

[symbol]

represents a single or double bond;
Ra represents an optionally substituted —NH-heterocycloalkyl radical;
Rb represents a hydrogen atom;
X represents S;
A represents S;
W represents a hydrogen atom; or the COR radical in which R represents:
a cycloalkyl radical, or an alkyl radical, optionally substituted with NR3R4 such that R3 and R4, which may be identical or different, represent a hydrogen atom or an alkyl radical;
   all the heterocycloalkyl radicals defined above being optionally substituted with one or more radicals chosen from halogen atoms, hydroxyl, oxo, alkoxy, NH₂. NHalk and N(alk)₂ radicals and alkyl and phenyl radicals which are themselves optionally substituted with one or more radicals chosen from halogen atoms and the radicals: hydroxyl, oxo, alkyl and alkoxy containing from 1 to 4 carbon atoms, NH₂. NHalk and N(alk)₂;
   said products of formula (I) being in any of the possible racemic, enantiomeric and diastereoisomeric isomer forms, and also the addition salts with mineral and organic acids or with mineral and organic bases of said products of formula (I).

A subject of the present invention is thus the products of formula (I) as defined above or below, corresponding to the following formulae:
3-[(2-amino-1,3-benzothiazol-6-yl)sulphanyl]-N-(tetrahydro-2H-pyran-4-yl)-[1,2,4]triazolo[4,3-b]pyridazin-6-amine
N-(6-{[6-(tetrahydro-2H-pyran-4-ylamino)[1,2,4]triazolo[4,3-b]pyridazin-3-yl]-sulphanyl}-1,3-benzothiazol-2-yl)acetamide
N-(6-{[6-(tetrahydro-2H-pyran-4-ylamino)[1,2,4]triazolo[4,3-b]pyridazin-3-yl]-sulphanyl}-1,3-benzothiazol-2-yl)cyclopropanecarboxamide
1-[2-(morpholin-4-yl)ethyl]-3-(6-{[6-(tetrahydro-2H-pyran-4-ylamino)[1,2,4]-triazolo[4,3-b]pyridazin-3-yl]sulphanyl}-1,3-benzothiazol-2-yl)urea
3-methoxy-N-(6-{[6-(tetrahydro-2H-pyran-4-ylamino)[1,2,4]triazolo[4,3-b]-pyridazin-3-yl]sulphanyl}-1,3-benzothiazol-2-yl)propanamide
N²,N²-dimethyl-N-(6-{[6-(tetrahydro-2H-pyran-4-ylamino)[1,2,4]triazolo-[4,3-b]pyridazin-3-yl]sulphanyl}-1,3-benzothiazol-2-yl)glycinamide
   and also the addition salts with mineral and organic acids or with mineral and organic bases of said products of formula (I).

A subject of the present invention is also any process for preparing the products of formula (I) as defined above.

A subject of the present invention is thus any process for preparing the products of formula (I) as defined above in which A represents NH.

A subject of the present invention is thus any process for preparing the products of formula (I) as defined above in which A represents S.

The products according to the invention can be prepared using conventional organic chemistry methods. Schemes 1, 2, 3, 4, 5 and 6 below illustrate the methods used for preparing the products of formula (I). In this respect, they cannot constitute a limitation of the scope of the invention, with regard to the methods for preparing the compounds claimed.

The products of formula (I) as defined above according to the present invention may thus in particular be prepared according to the processes described in schemes 1, 2, 3, 4, 5 and 6 below.

A subject of the present invention is thus also the process for preparing products of formula (I) according to scheme 1 as defined hereinafter

A subject of the present invention is thus also the process for preparing products of formula (I) according to scheme 2 as defined hereinafter.

A subject of the present invention is thus also the process for preparing products of formula (I) according to scheme 3 as defined hereinafter.

A subject of the present invention is thus also the process for preparing products of formula (I) according to scheme 4 as defined hereinafter.

A subject of the present invention is thus also the process for preparing products of formula (I) according to scheme 5 as defined hereinafter.

A subject of the present invention is thus also the process for preparing products of formula (I) according to scheme 6 as defined hereinafter.

Just as, among the products of formula (I) as defined above in which [symbol] represents a single or double bond, the products of formula (I′) which represent the products of formula (I) in which [symbol] represents a single bond and the products of formula (I″) which represent the products of formula (I) in which [symbol] represents a double bond are defined,

similarly, for the synthesis intermediates as defined hereinafter, of formulae (a), (b), (c), (d), (e) and (f) in which [symbol] represents a single or double bond, the compounds of formulae (a′), (b′), (c′), (d′), (e′) and (f′) in which [symbol] represents a single bond, and the compounds of formulae (a″), (b″), (c″), (d″), (e″) and (f″) in which [symbol] represents a double bond are defined.

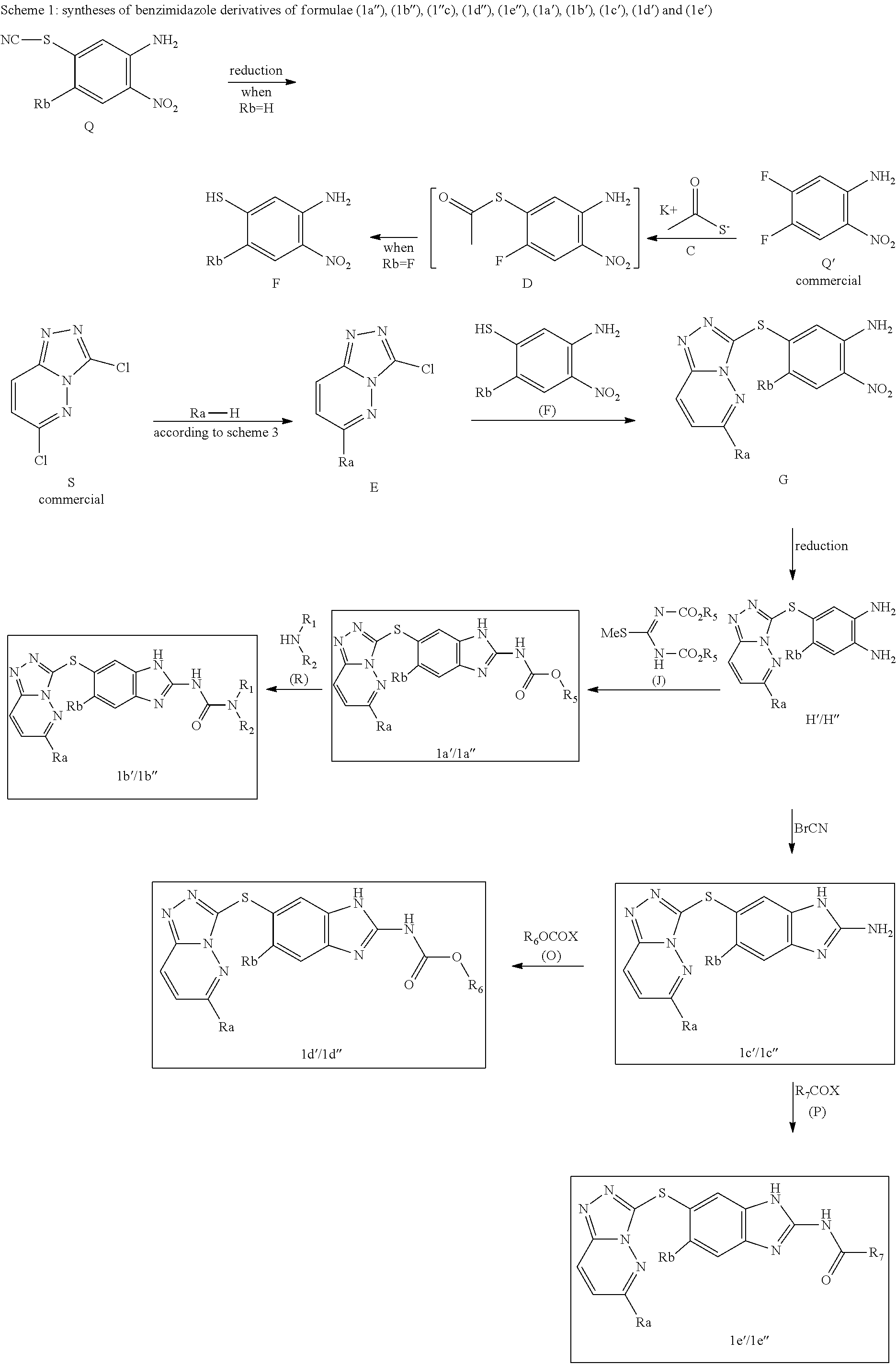

In scheme 1 above, the substituents Ra and Rb have the meanings indicated above for the products of formulae (I′) and (I″). The substituent R5, in the compounds of formulae (J), (1a′) and (1a″), represents an alkyl radical.

In scheme 1 above, the CONR1R2, CO₂R6 and COR7 groups, which constitute W, can take the values of W as defined above for the products of formula (I′) and (I″), when W≠H

In above scheme 1, the benzimidazoles of general formulae (1a″), (1b″), (1c″), (1d″) and (1e″) and also the reduced analogues thereof of general formulae (1a′), (1b′), (1c′), (1d′) and (1e′) can be prepared from commercial 3,6-dichloro[1,2,4]triazolo[4,3-b]pyridazine of formula (S)

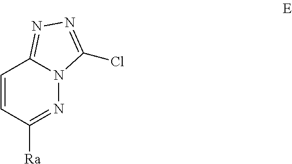

The compounds (E) can be obtained, for example, by reaction of amines with the compound (S). The reaction is carried out, for example, at a temperature in the region of 20 to 50° C.

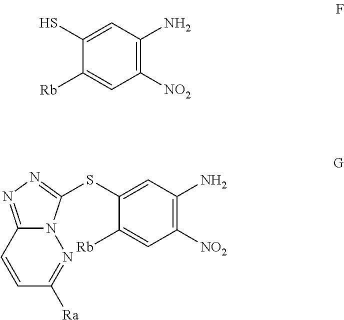

The compounds (G) with Rb=H can be obtained, for example, by reaction of 3-amino-4-nitrobenzenethiol of formula (F) with the compounds of formula (E). The compounds of formula (F) are obtained by reduction, in situ, of 3-amino-4-nitrophenyl thiocyanate (Q) (commercial compound), for example, in the presence of sodium borohydride in a solvent such as N,N-dimethylformamide, at a temperature in the region of 20° C.

The compounds (G) with Rb=F can be obtained, for example, by reaction of 2-fluoro-5-amino-4-nitrobenzenethiol of formula (F) with the compounds of formula (E). The compounds of formula (F) with Rb=F are obtained by reaction of 2-nitro-4,5-difluoroaniline (Q′) (commercial compound), for example, in the presence of potassium thioacetate (C) in a solvent such as N,N-dimethylformamide, at a temperature in the region of 20° C.

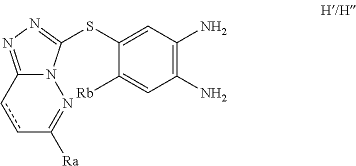

The compounds (H″) such that [symbol] represents a double bond can be obtained, for example, by reduction with iron (0) on the compounds of formula (G), in a solvent such as methanol, in the presence of acetic acid, at a temperature in the region of 70° C.

The compounds (H′) such that [symbol] represents a single bond can be obtained, for example, by reduction with zinc (0) on the compounds of formula (G), in the presence of acetic acid, at a temperature in the region of 20° C.

More particularly, the carbamates of general formulae (1a′) and (1a″) can be prepared in particular as described in patent WO03028721A2, but using respectively a 3,4-diaminophenyl sulphide of formulae (H′) and (H″) and a pseudo thiourea of formula (J), in the presence of acetic acid and in a protic solvent such as methanol, at a temperature in the region of 80° C.

More particularly, the benzimidazoles of general formulae (1b′) and (1b″) can be prepared respectively by reaction of an amine NHR1R2 of formula (R) (with R1 and R2 as defined above) with a carbamate of formulae (1a′) and (1a″), for example in the presence of an aprotic solvent such as 1-methyl-2-pyrrolidinone. The reaction is carried out, for example, at a temperature in the region of 120° C., in a sealed tube under microwaves.

More particularly, the 2-amino benzimidazoles of general formulae (1c′) and (1c″) can be prepared, for example, by reaction of cyanogen bromide with a compound of formulae respectively (H′) and (H″), in the presence of a protic solvent such as ethanol. The reaction is carried out at a temperature in the region of 80° C.

More particularly, the general carbamates of formulae (1d′) and (1d″) can be obtained by reaction with a chlorocarbonate of formula (O) (X═Cl) on a compound of general formulae respectively (1c′) and (1c″), for example in a solvent such as tetrahydrofuran, in the presence of a base such as sodium hydrogen carbonate at a temperature in the region of 20° C.

More particularly, the carboxamides (1e′) and (1e″) can be obtained respectively from the amines of general formulae (1c′) and (1c″)
by reaction of the amines (1c′) and (1c″) with an acid chloride of formula (P) (X═Cl), in the presence, for example, of a solvent such as pyridine, at a temperature in the region of 20° C.
by reaction of the amines (1c′) and (1c″) with an acid anhydride of formula (P) (X═OCOR7), in the presence, for example, of a solvent such as pyridine at a temperature in the region of 20° C.
by coupling of the amines (1c′) and (1c″) with an acid of formula (P) (X═OH) under the conditions described, for example, by D. D. DesMarteau; V. Montanari (Chem Lett, 2000 (9), 1052), in the presence of 1-hydroxybenzotriazole and 1-(3-dimethylaminopropyl)-3-ethylcarbodiimide and in the presence of a base such as triethylamine, at a temperature in the region of 40° C.

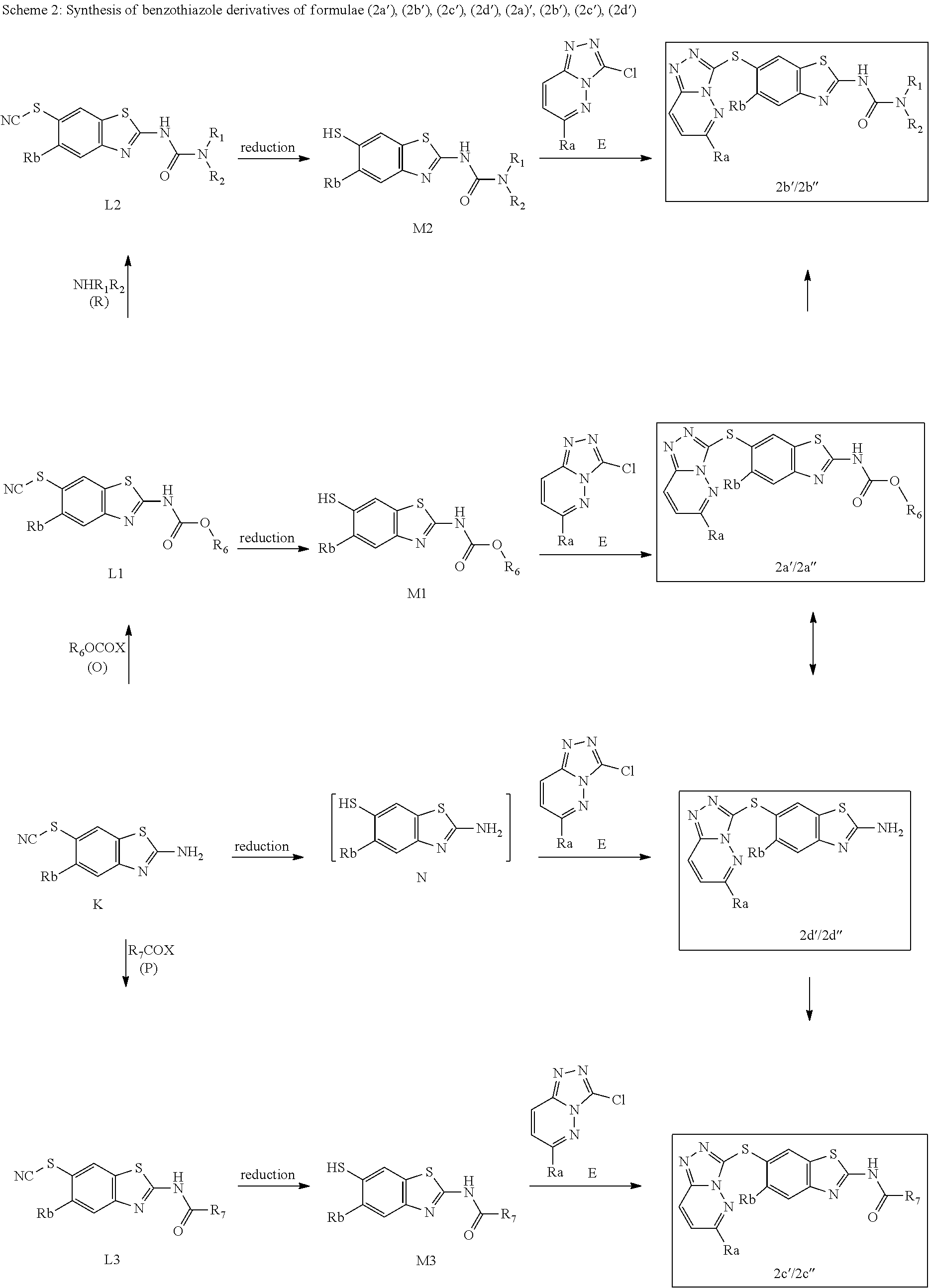

In scheme 2 above, the substituents Ra and Rb have the meanings indicated above for the products of formulae (I′) and (I″). Similarly, the CONR1R2, CO₂R6 and COR7 groups, which constitute W, can take the values of W as defined above for the products of formula (I′) and (I″), when W≠H.

In scheme 2 above, the benzothiazoles of general formulae (2a″), (2b″), (2c″) and (2d″) and also the reduced analogues thereof of general formulae (2a′), (2b′), (2c′) and (2d′) can be prepared from 2-amino-1,3-benzothiazol-6-yl thiocyanate (K) with Rb=H (commercial compound).

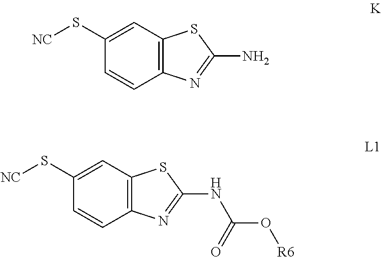

The carbamates of general formula (L1) can be obtained, for example, by reaction with a chlorocarbonate of formula (O) (X═Cl) on 2-amino-1,3-benzothiazol-6-yl thiocyanate with Rb=H (K), in a solvent such as tetrahydrofuran, in the presence of a base such as sodium hydrogen carbonate, at a temperature in the region of 20° C.

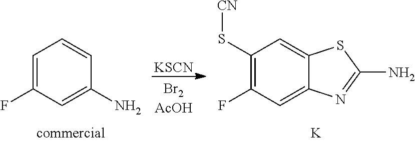

In the above scheme 2, the benzothiazoles of general formulae (2a″), (2b″), (2c″) and (2d″) and also the reduced analogues thereof of general formulae (2a′), (2b′), (2c′) and (2d′) with Rb=F can be prepared from 2-amino-5-fluoro-1,3-benzothiazol-6-yl thiocyanate. The 2-amino-5-fluoro-1,3-benzothiazol-6-yl thiocyanate (K) can be prepared in the manner described by K. Papke and R. Pohloudek-Fabini in Pharmazie; GE; 22, 5 1967, P229-233, by reaction of potassium thiocyanate and 3-fluoroaniline in the presence of bromine in acetic acid.

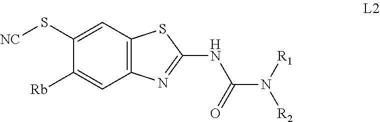

The compounds of general formula (L2) can be obtained, for example, by reaction of the carbamates of formula (L1) where R6=phenyl, with amines NHR1R2 of formula (R) (with Rb, R1 and R2 as defined above), in the presence of an aprotic solvent such as tetrahydrofuran, at a temperature in the region of 20° C.

The ureas (2b′) and (2b″) can be obtained, for example, respectively from the carbamates (2a′) and (2a″) where R6=phenyl, in the same way as the ureas (L2) are obtained by reaction of amines with the carbamates of type (L1).

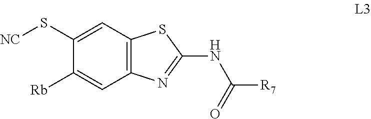

The compounds of general formula (L3) can be obtained, for example:
by reaction of an acid chloride of formula (P) (X═Cl) with 2-amino-1,3-benzothiazol-6-yl thiocyanate (K), in the presence, for example, of a solvent such as pyridine, at a temperature in the region of 20° C.
by reaction of an acid anhydride of formula (P) (X═OCOR7) with 2-amino-1,3-benzothiazol-6-yl thiocyanate (K), in the presence, for example, of a solvent such as pyridine, at a temperature in the region of 20° C.
by coupling of 2-amino-1,3-benzothiazol-6-yl thiocyanate (K) with an acid of formula (P) (X═OH) under the conditions described, for example, by D. D. DesMarteau; V. Montanari (Chem Lett, 2000 (9), 1052), in the presence of 1-hydroxybenzotriazole and 1-(3-dimethylaminopropyl)-3-ethylcarbodiimide and in the presence of a base such as triethylamine, at a temperature in the region of 40° C.

In the same way as the carboxamides (L3) can be obtained by acylation of the amine (K), the carboxamides (2c′) and (2c″) can be obtained respectively from the amines (2d′) and (2d″).

The compounds of general formulae (M1), (M2) and (M3) can be obtained, for example, by reduction of compounds of general formulae (L1), (L2), (L3) with DL-dithiothreitol, in the presence of sodium dihydrogen carbonate, in a solvent such as ethanol and at a temperature in the region of 80° C.

The compound of general formula (N) can be prepared in situ by reduction of the compound of formula (K), for example with sodium borohydride in a solvent such as N,N-dimethylformamide, in the presence of a base such as triethylamine and at a temperature in the region of 95° C. or between 20° C. and 95° C.

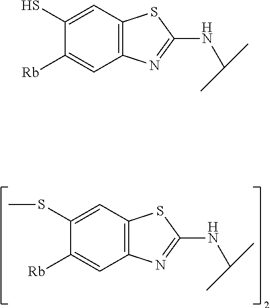

The aryl-thiol intermediates above can exist in the form of free thiols or in the form of disulphides or a mixture of the two forms which can be employed without distinction in the subsequent reactions.

More particularly, the benzothiazoles of general formulae (2d′) and (2d″) can also be prepared respectively from carbamates of formulae (2a′) and (2a″) where R6=t-butyl, by reaction, for example, with trifluoroacetic acid in a solvent such as dichloromethane, at a temperature in the region of 20° C.

Reciprocally, the benzothiazoles of general formulae (2a′) and (2a″) can also be prepared from benzothiazoles of formulae respectively (2d′) and (2d″), for example by reaction with a chlorocarbonate of formula (O) (X═Cl), in a solvent such as tetrahydrofuran, in the presence of a base such as sodium hydrogen carbonate, at a temperature in the region of 20° C.

More particularly, the benzothiazoles of general formulae (2a″), (2b″), (2c″) and (2d″) and also the reduced analogues thereof of general formulae (2a′), (2b′), (2c′) and (2d′) can be prepared, for example:
1) either by coupling of a compound of formula (E) with derivatives (M1), (M2) and (M3) and (N) generated in situ by reduction of the derivatives (L1), (L2), (L3) and (K) with sodium borohydride, in a solvent such as N,N-dimethylformamide and in the presence of a base such as triethylamine, at a temperature in the region of 95° C. or else between 50° C. and 95° C.
2) or by coupling of the isolated derivatives (M1), (M2) and (M3) and of a compound of formula (E), in the presence of sodium borohydride in a solvent such as N,N-dimethylformamide and in the presence of a base such as triethylamine, at a temperature in the region of 95° C.
3) or by coupling of a compound of formula (E) with derivatives (M1), (M2) and (M3) and (N) generated in situ by reduction of the derivatives (L1), (L2), (L3) and (K) in the presence of DL-dithiothreitol and of sodium dihydrogen carbonate, in a solvent such as ethanol and at a temperature in the region of 80° C.

The reducing conditions 1) and 2) can give products of formulae (2a), (2b), (2c) and (2d) such that [symbol] represents a single or double bond, whereas the conditions 3) and 4) give products of formula (2a), (2b), (2c) and (2d) such that [symbol] represents a double bond.

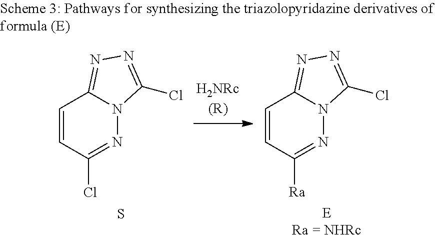

In scheme 3 above, the substituents Ra and Rc have the meanings indicated above for the products of formulae (I′) and (I″).

The compounds of formula (E) can be obtained, for example, as indicated in scheme 3 above, from commercial 3,6-dichloro[1,2,4]triazolo[4,3-b]pyridazine of formula (S).

More particularly, the compounds of formula (E) where Ra represents an NHRc radical can be obtained by treatment of 3,6-dichloro[1,2,4]-triazolo[4,3-b]pyridazine (S) with an amine of formula RcNH₂, at a temperature in the region of 20° C. and in a solvent such as N,N-dimethylformamide at a temperature of between 20° and 50° C.

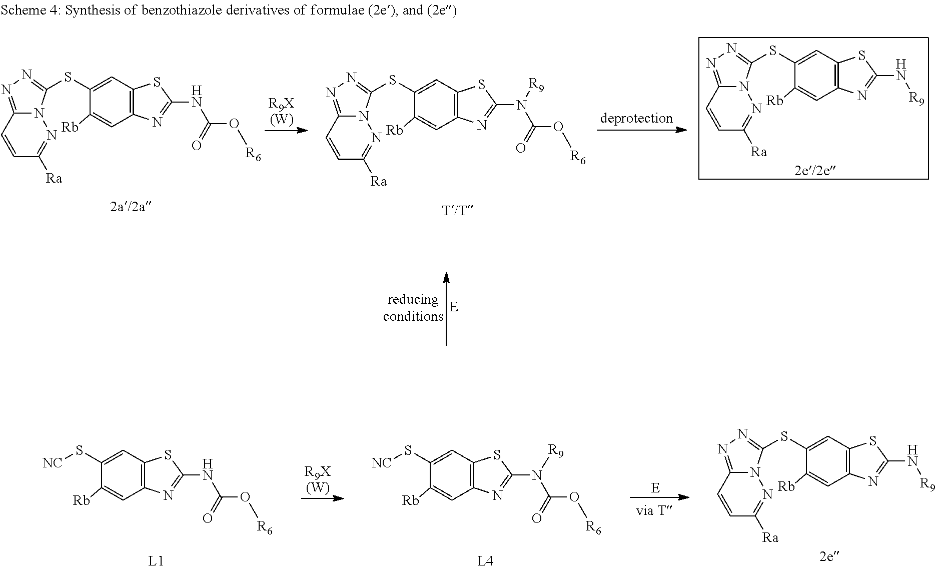

According to scheme 4 above, the benzothiazoles of general formulae (2e′) and (2e″) can be prepared respectively from the compounds of formulae (2a′) and (2a″).

In scheme 4 above, the substituent OR6 preferably represents O-t-butyl. The substituent R9 represents an alkyl or cycloalkyl radical optionally substituted with an alkoxy, heterocycloalkyl or NR3R4 radical (R3 and R4 as defined above).

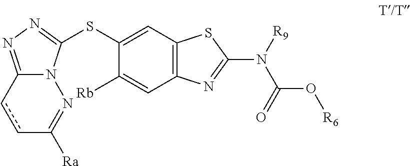

The carbamates of general formula (T′) and (T″) can be obtained respectively by reaction of carbamates of general formulae (2a′) and (2a″) with R6=tBu preferably, for example, with alkyl halides of formula (W), in a solvent such as N,N-dimethylformamide, in the presence of sodium hydride, at a temperature of between 20 and 90° C.

The benzothiazoles of general formula (2e′) and (2e″) can also be prepared from the compounds of formula (L1), preferably with R6=tBu, via the compounds of formulae (T′) and (T″).

More particularly, the compounds of general formulae (2e′) and (2e″) can be obtained respectively by treatment of the isolated compounds (T′) and (T″), for example, with trifluoroacetic acid, in a solvent such as dichloromethane, at a temperature in the region of 20° C.

Alternatively, the compounds of general formula (2e″) can be obtained directly by reaction of the compounds of formulae (L4) and (E), via the compound (T″) formed in situ, for example, in the presence of 131, dithiothreitol and sodium dihydrogen carbonate, in a solvent such as ethanol and at a temperature in the region of 80° C., optionally followed by a treatment in situ with trifluoroacetic acid at 20° C. if necessary.

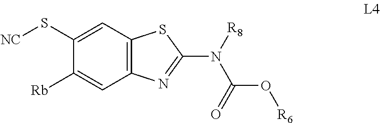

The carbamates of general formula (L4) can be obtained by reaction of carbamates of general formula (L1), for example with alkyl halides of formula (W), in a solvent such as N,N-dimethylformamide, in the presence of sodium hydride, at a temperature of between 20 and 90° C.

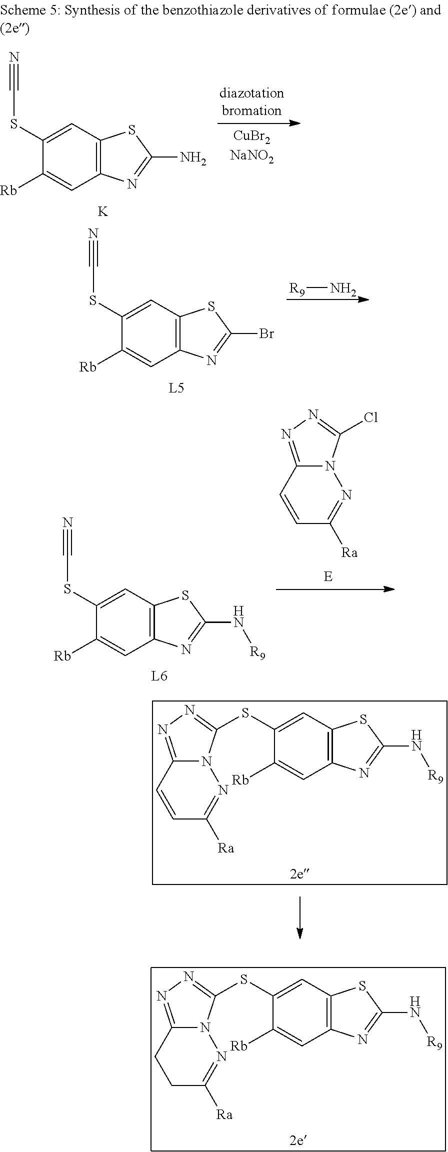

Alternatively, according to scheme 5 above, the benzothiazoles of general formula (2e″) can be prepared from the compounds of formulae (L6) and (E), for example in the presence of DL-dithiothreitol and sodium dihydrogen carbonate, in a solvent such as ethanol and at a temperature in the region of 80° C.

The benzothiazoles of general formula (2e′) can be prepared from the compounds of formula (2e″), according to the methods described below for preparing the compounds (I′) from the compounds (I″).

The compounds of formula (L6) can be prepared from the 2-bromobenzothiazole derivative (L5) by treatment with an NH2R9 derivative, for example, in a solvent such as tetrahydrofuran, at a temperature in the region of 20° C.

The substituent R9 represents an alkyl or cycloalkyl radical optionally substituted with an alkoxy, heterocycloalkyl or NR3R4 radical (R3 and R4 as defined above).

The compounds of formula (L5) can be prepared from 2-amino-1,3-benzothiazol-6-yl thiocyanate (K) (commercial compound), for example, by treatment with an alkyl nitrite and cuprous bromide in a solvent such as acetonitrile, at a temperature in the region of 0-20° C., according to the method described by Jagabandhu Das et. al., in J. Med. Chem. 2006, 49, 6819-6832.

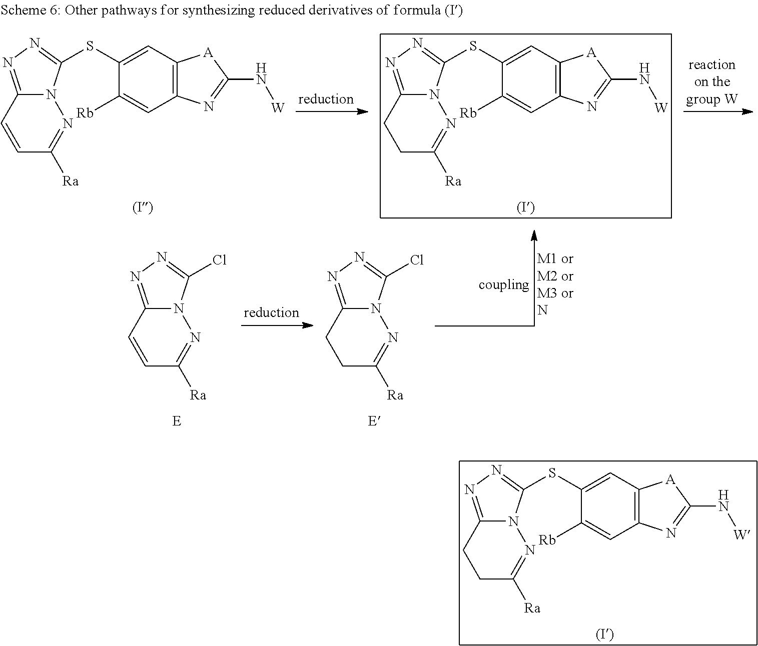

According to scheme 6 above, the benzothiazoles of general formula (I′) can also be prepared, from the compounds of formula (I″), by reduction, for example, with sodium borohydride, in a solvent such as ethanol, at a temperature in the region of 80° C., or else by reduction with zinc(0) in the presence of acetic acid, at a temperature in the region of 20° C.

Alternatively, the compounds (I′) can also be prepared from the compounds of formula (E′) by coupling with the compounds of type M1, M2, M3 or N, obtained as intermediates by reduction of the compounds L1, L2, L3 or K in situ, as described above in scheme 2. The compounds of type M1, M2 or M3 can also be isolated and used for the coupling with (E′). The compounds (E′) can be obtained from the compounds of formula (E) by reduction, for example by reduction with zinc(0) in the presence of acetic acid, at a temperature in the region of 20° C.

Alternatively, the compounds (I′) can also be prepared from other compounds (I′) by conversion of the group W to a group W′ of the same nature as defined above for W and according to reactions of the type defined in scheme 2: conversions of 2d′/2d″ to 2a′/2a″ and to 2c′/2c″, conversions of 2a′/2a″ to 2d′/2d″ and to 2b′/2b″.

In the compounds of general formula (I) as defined above, the sulphur S can be oxidized to sulphoxide SO or sulphone SO₂ according to the methods known to those skilled in the art and while protecting, if necessary, the possibly reactive groups with appropriate protecting groups.

Among the starting products of formulae J, K, O, P, Q, Q′, R, S, U and W, some are known and can be obtained either commercially, or according to the usual methods known to those skilled in the art, for example starting from commercial products.

It is understood, for those skilled in the art, that, in order to carry out the processes according to the invention described above, it may be necessary to introduce protecting groups for amino, carboxyl and alcohol functions in order to avoid side reactions.

The following non-exhaustive list of examples of protection of reactive functions may be mentioned:
hydroxyl groups may be protected, for example, with alkyl radicals such as tert-butyl, trimethylsilyl, tert-butyldimethylsilyl, methoxymethyl, tetrahydropyranyl, benzyl or acetyl,
amino groups may be protected, for example, with acetyl, trityl, benzyl, tertbutoxycarbonyl, BOC, benzyloxycarbonyl or phthalimido radicals or other radicals known in peptide chemistry.

Acid functions may be protected, for example, in the form of esters formed with readily cleavable esters such as benzyl or tert-butyl esters or esters known in peptide chemistry.

A list of various protecting groups that may be used will be found in the textbooks known to those skilled in the art and, for example, in patent BF 2 499 995.

It may be noted that it is possible, if desired and if necessary, to subject intermediate products or products of formula (I) thus obtained by the processes indicated above, in order to obtain other intermediates or other products of formula (I), to one or more conversion reactions known to those skilled in the art, for instance:

a) a reaction for esterification of an acid function,
b) a reaction for saponification of an ester function to an acid function,
c) a reaction for reducing a free or esterified carboxyl function to an alcohol function,
d) a reaction for conversion of an alkoxy function to a hydroxyl function, or alternatively of a hydroxyl function to an alkoxy function,
e) a reaction for removal of the protecting groups that may be borne by the protected reactive functions,
f) a reaction for salification with a mineral or organic acid or with a base so as to obtain the corresponding salt,
g) a reaction for resolution of the racemic forms to resolved products,
said products of formula (I) thus obtained being in any of the possible racemic, enantiomeric and diastereoisomeric isomer forms.

The reactions a) to g) can be carried out under the usual conditions known to those skilled in the art, for instance those indicated hereinafter.

a) The products described above may, if desired, undergo, on the possible carboxyl functions, esterification reactions that may be performed according to the usual methods known to those skilled in the art.

b) The possible conversions of ester functions to acid functions of the products described above may be performed, if desired, under the usual conditions known to those skilled in the art, in particular by acid or alkaline hydrolysis, for example with sodium hydroxide or potassium hydroxide in alcoholic medium, for instance in methanol, or alternatively with hydrochloric acid or sulphuric acid.

The saponification reaction may be performed according to the usual methods known to those skilled in the art, for instance in a solvent such as methanol or ethanol, dioxane or dimethoxyethane, in the presence of sodium hydroxide or potassium hydroxide.

c) The possible free or esterified carboxyl functions of the products described above may be reduced, if desired, to alcohol functions via the methods known to those skilled in the art: the possible esterified carboxyl functions may be reduced, if desired, to alcohol functions by the methods known to those skilled in the art, and in particular with lithium aluminium hydride in a solvent, for instance tetrahydrofuran, or dioxane or ethyl ether.

The possible free carboxyl functions of the products described above may be reduced, if desired, to alcohol functions, in particular with boron hydride.

d) The possible alkoxy functions, such as in particular methoxy, of the products described above may be converted, if desired, into hydroxyl functions under the usual conditions known to those skilled in the art, for example with boron tribromide in a solvent such as, for example, methylene chloride, with pyridine hydrochloride or hydrobromide, or alternatively with hydrobromic or hydrochloric acid in water or trifluoroacetic acid at reflux.

e) The removal of protecting groups, for instance those indicated above, may be performed under the usual conditions known to those skilled in the art, in particular via an acid hydrolysis performed with an acid such as hydrochloric acid, benzenesulphonic acid or para-toluenesulphonic acid, formic acid or trifluoroacetic acid, or alternatively via catalytic hydrogenation.

The phthalimido group may be removed with hydrazine.

f) The products described above may undergo, if desired, salification reactions, for example with a mineral or organic acid or with a mineral or organic base according to the usual methods known to those skilled in the art: such a salification reaction may be performed, for example, in the presence of hydrochloric acid, or alternatively of tartaric acid, citric acid or methanesulphonic acid, in an alcohol, for instance ethanol or methanol.

g) The possible optically active forms of the products described above may be prepared by resolution of the racemic mixtures according to the usual methods known to those skilled in the art.

The products of formula (I) as defined above and also the addition salts thereof with acids show advantageous pharmacological properties, in particular on account of their kinase-inhibiting properties as indicated above.

The products of the present invention can in particular be used for treating tumours.

The products of the invention may thus also increase the therapeutic effects of commonly used antitumour agents.

These properties justify their therapeutic use, and a subject of the invention is in particular, as medicaments, the products of formula (I) as defined above, said products of formula (I) being in any of the possible racemic, enantiomeric and diastereoisomeric isomer forms, and also the addition salts with pharmaceutically acceptable mineral and organic acids or with pharmaceutically acceptable mineral and organic bases of said products of formula (I).

A subject of the invention is most particularly, as medicaments, the products corresponding to the following formulae:
3-[(2-amino-1,3-benzothiazol-6-yl)sulphanyl]-N-(tetrahydro-2H-pyran-4-yl)-[1,2,4]triazolo[4,3-b]pyridazin-6-amine
N-(6-{[6-(tetrahydro-2H-pyran-4-ylamino)[1,2,4]triazolo[4,3-b]pyridazin-3-yl]-sulphanyl}-1,3-benzothiazol-2-yl)acetamide
N-(6-{[6-(tetrahydro-2H-pyran-4-ylamino)[1,2,4]triazolo[4,3-b]pyridazin-3-yl]-sulphanyl}-1,3-benzothiazol-2-yl)cyclopropanecarboxamide
1-[2-(morpholin-4-yl)ethyl]-3-(6-{[6-(tetrahydro-2H-pyran-4-ylamino)[1,2,4]-triazolo[4,3-b]pyridazin-3-yl]sulphanyl}-1,3-benzothiazol-2-yl)urea
3-methoxy-N-(6-{[6-(tetrahydro-2H-pyran-4-ylamino)[1,2,4]triazolo[4,3-b]-pyridazin-3-yl]sulphanyl}-1,3-benzothiazol-2-yl)propanamide
N²,N²-dimethyl-N-(6-{[6-(tetrahydro-2H-pyran-4-ylamino)[1,2,4]triazolo-[4,3-b]pyridazin-3-yl]sulphanyl}-1,3-benzothiazol-2-yl)glycinamide
   and also the addition salts with pharmaceutically acceptable mineral and organic acids or with pharmaceutically acceptable mineral and organic bases of said products of formula (I).

The invention also relates to pharmaceutical compositions containing, as active ingredient, at least one of the products of formula (I) as defined above or a pharmaceutically acceptable salt of this product or a prodrug of this product and, where appropriate, a pharmaceutically acceptable carrier.

The invention thus covers the pharmaceutical compositions containing, as active ingredient, at least one of the medicaments as defined above.

Such pharmaceutical compositions of the present invention may also, where appropriate, contain active ingredients of other antimitotic medicaments, such as in particular those based on taxol, cisplatin, DNA intercalating agents, and the like.

These pharmaceutical compositions may be administered orally, parenterally or locally by topical application to the skin and the mucous membranes or by intravenous or intramuscular injection.

These compositions may be solid or liquid and may be in any pharmaceutical form commonly used in human medicine, for instance simple or sugar-coated tablets, pills, lozenges, gel capsules, drops, granules, injectable preparations, ointments, creams or gels; they are prepared according to the usual methods. The active ingredient may be incorporated therein into excipients normally used in these pharmaceutical compositions, such as talc, gum arabic, lactose, starch, magnesium stearate, cocoa butter, aqueous or nonaqueous carriers, fatty substances of animal or plant origin, paraffinic derivatives, glycols, various wetting agents, dispersants or emulsifiers, and preservatives.

The usual dosage, which is variable depending on the product used, the individual treated and the condition in question, may be, for example, from 0.05 to 5 g per day in adults, or preferably from 0.1 to 2 g per day.

A subject of the invention is also the use of the products of formula (I) as defined above or of pharmaceutically acceptable salts of these products for the preparation of a medicament for use in inhibiting the activity of a protein kinase.

A subject of the present invention is also the use of products of formula (I) as defined above for the preparation of a medicament for use in the treatment or prevention of a disease characterized by deregulation of the activity of a protein kinase.

Such a medicament may in particular be for use in the treatment or prevention of a disease in a mammal.

A subject of the present invention is also the use defined above, in which the protein kinase is a protein tyrosine kinase.

A subject of the present invention is also the use defined above, in which the protein tyrosine kinase is MET or mutant forms thereof.

A subject of the present invention is also the use defined above, in which the protein kinase is in a cell culture.

A subject of the present invention is also the use defined above, in which the protein kinase is in a mammal.

A subject of the present invention is in particular the use of a product of formula (I) as defined above for the preparation of a medicament for use in the prevention or treatment of diseases associated with an uncontrolled proliferation.

A subject of the present invention is in particular the use of a product of formula (I) as defined above for the preparation of a medicament for use in the treatment or prevention of a disease chosen from the following group: blood vessel proliferation disorders, fibrotic disorders, ‘mesangial’ cell proliferation disorders, metabolic disorders, allergies, asthma, thrombosis, nervous system diseases, retinopathy, psoriasis, rheumatoid arthritis, diabetes, muscle degeneration and cancers.

A subject of the present invention is thus most particularly the use of a product of formula (I) as defined above for the preparation of a medicament for use in the treatment or prevention of diseases in oncology and in particular for use in the treatment of cancers.

Among these cancers, the treatment of solid or liquid tumours and the treatment of cancers that are resistant to cytotoxic agents are of interest.

The cited products of the present invention may in particular be used for the treatment of primary tumours and/or metastases, in particular in gastric, hepatic, renal, ovarian, colon, prostate and lung (NSCLC and SCLC) cancers, glioblastomas, thyroid, bladder or breast cancers, in melanomas, in lymphoid or myeloid haematopoietic tumours, in sarcomas, in brain, larynx or lymphatic system cancers, bone cancers and pancreatic cancers.

A subject of the present invention is also the use of the products of formula (I) as defined above for the preparation of medicaments for use in cancer chemotherapy.

Such medicaments for use in cancer chemotherapy may be used alone or in combination.

The products of the present application may in particular be administered alone or in combination with chemotherapy or radiotherapy or alternatively in combination, for example, with other therapeutic agents.

Such therapeutic agents may be commonly used antitumour agents.

As kinase inhibitors, mention may be made of butyrolactone, flavopiridol and 2(2-hydroxyethylamino)-6-benzylamino-9-methylpurine, also known as olomucine.

A subject of the present invention is also, as new industrial products, the synthesis intermediates of formulae Mt M2, M3 and N as defined above and recalled hereinafter:

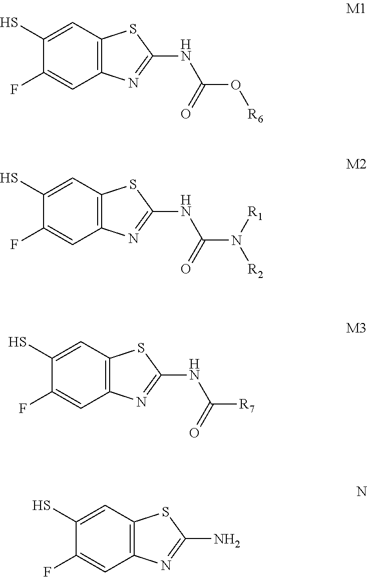

in which the CONR1R2, CO₂R6 and COR7 groups, which constitute W, can take the values of W as defined above for the products of formula (I′) and (I″), when W≠H.

The following examples, which are products of formula (I), illustrate the invention without, however, limiting it.

### EXPERIMENTAL SECTION

The nomenclature of the compounds of the present invention was carried out with the ACDLABS software version 11.0.

Microwave oven used:

Biotage, Initiator EXP-EU, 300 W max, 2450 MHz

The ¹H NMR spectrum at 400 MHz and ¹H NMR spectrum at 300 MHz were acquired on a Bruker Avance DRX-400 or Bruker Avance DPX-300 spectrometer with the chemical shifts (δ in ppm) in the solvent d6-dimethyl sulphoxide (d6-DMSO) referenced to 2.5 ppm at a temperature of 303 K.

The mass spectra were acquired either by:
LC-MS-DAD-ELSD analysis (MS=Waters ZQ), or
LC-MS-DAD-ELSD analysis (MS=Platform II Waters Micromass) or
HPLC-MS-DAD-ELSD analysis (MS=Quattro Premier XE Waters).

DAD considered wavelength λ=210-400 nm
ELSD: Sedere SEDEX 85; nebulization temperature=35° C.; nebulization pressure=3.7 bar.

### Example 1

### 3-[(2-Amino-1,3-benzothiazol-6-yl)sulphanyl]-N-(tetrahydro-2H-pyran-4-yl)[1,2,4]triazolo[4,3-b]pyridazin-6-amine

a) The 3-[(2-amino-1,3-benzothiazol-6-yl)sulphanyl]-N-(tetrahydro-2H-pyran-4-yl)[1,2,4]triazolo[4,3-b]pyridazin-6-amine can be prepared in the following way:

a stream of argon is sparged, for 5 minutes, through a mixture of 900 mg of 2-amino-1,3-benzothiazol-6-yl thiocyanate in 27 cm³ of ethanol. 21 mg of potassium dihydrogen phosphate in 2.7 cm³ of water, 2.01 g of DL-dithiothreitol and 1.21 g of 3-chloro-N-(tetrahydro-2H-pyran-4-yl)[1,2,4]-triazolo[4,3-b]pyridazin-6-amine are subsequently added. The reaction mixture is heated at 80° C. for 18 h. The suspension obtained is cooled to 20° C. and the precipitate is spin-filter-dried and then washed with water. The solid obtained, in hydrochloride form, is taken up in a mixture of 10 cm³ of water and 5 cm³ of 1N sodium hydroxide. The suspension is stirred for 10 min at 20° C. and then the solid is spin-filter-dried, washed with water and dried under vacuum. 1.32 g of 3-[(2-amino-1,3-benzothiazol-6-yl)sulphanyl]-N-(tetrahydro-2H-pyran-4-yl)[1,2,4]triazolo[4,3-b]pyridazin-6-amine are thus obtained in the form of a creamy-white powder, the characteristics of which are as follows:

1H NMR SPECTRUM (400 MHz, DMSO-d6) δ.ppm 1.23-1.44 (m, 2H) 1.77 (d, J=12.7 Hz, 2H) 3.35 (t, J=11.5 Hz, 2H) 3.53-3.71 (m, 1H) 3.79 (d, J=11.7 Hz, 2H) 6.78 (d, J=9.8 Hz, 1H) 7.12-7.32 (m, 2H) 7.37 (d, J=6.8 Hz, 1H) 7.59 (s, 2H) 7.78 (d, J=1.2 Hz, 1H) 7.93 (d, J=9.8 Hz, 1H)

MASS SPECTRUM: Waters HPLC-SQD: MH+m/z=400; MH−=398−

b) The 3-chloro-N-(tetrahydro-2H-pyran-4-yl)[1,2,4]triazolo[4,3-b]-pyridazin-6-amine can be prepared in the following way:

2.1 cm³ of tetrahydro-2H-pyran-4-amine and 3 cm³ of triethylamine are added to a solution of 2 g of commercial 3,6-dichloro[1,2,4]triazolo[4,3-b]pyridazine in 20 cm³ of N,N-dimethylformamide. The reaction mixture is stirred at 20° C. for 18 h and 3 h at 50° C., and then cooled to 20° C., before the addition of 20 cm³ of water. The white precipitate is spin-filter-dried and then washed successively with water and ether. 1.3 g of 3-chloro-N-(tetrahydro-2H-pyran-4-yl)[1,2,4]triazolo[4,3-b]pyridazin-6-amine are thus obtained in the form of a white powder, the characteristics of which are as follows:

MASS SPECTRUM: Waters ZQ: MH+m/z=254+; MH−=252−

### Example 2

### N-(6-{[6-(Tetrahydro-2H-pyran-4-ylamino)[1,2,4]triazolo[4,3-b]pyridazin-3-yl]sulphanyl}-1,3-benzothiazol-2-yl)acetamide

a) The N-(6-{(6-(tetrahydro-2H-pyran-4-ylamino)[1,2,4]triazolo[4,3-N-pyridazin-3-yl]sulphanyl}-1,3-benzothiazol-2-yl)acetamide can be prepared in the following way:

0.212 cm³ of acetyl chloride is added to 300 mg of 3-[(2-amino-1,3-benzothiazol-6-yl)sulphanyl]-N-(tetrahydro-2H-pyran-4-yl)[1,2,4]triazolo-[4,3-b]pyridazin-6-amine (1a) and 0.42 cm³ of triethylamine in 6 cm³ of dichloromethane at 20° C. After 20 h, the reaction mixture is concentrated to dryness and the solid residue is chromatographed by solid deposit on Biotage Quad 12/25 (KP-SIL, 60A; 32-63 μM), elution being carried out with a dichloromethane/(38 dichloromethane/17 methanol/2 aqueous ammonia) gradient of 95/5 to 70/30. 176 mg of N-(6-{[6-(tetrahydro-2H-pyran-4-ylamino)[1,2,4]triazolo[4,3-b]pyridazin-3-yl]sulphanyl}-1,3-benzothiazol-2-yl)acetamide are thus obtained in the form of a beige powder, the characteristics of which are as follows:

1H NMR SPECTRUM (400 MHz, DMSO-d6) δ.ppm 1.14-1.37 (m, 2H) 1.66 (d, J=11.0 Hz, 2H) 2.19 (s, 3H) 3.19-3.27 (m, 2H) 3.45-3.62 (m, 1H) 3.69 (d, J=11.5 Hz, 2H) 6.79 (d, J=10.0 Hz, 1H) 7.15-7.46 (m, 2H) 7.64 (d, J=8.6 Hz, 1H) 7.95 (d, J=10.0 Hz, 1H) 8.04 (d, J=1.7 Hz, 1H) 12.37 (br. s., 1H)

MASS SPECTRUM: Waters HPLC-SQD: MH+m/z=442+; MH−=440−

### Example 3

### N-(6-{[6-(Tetrahydro-2H-pyran-4-ylamino)[1,2,4]triazolo[4,3-b]pyridazin-3-yl]sulphanyl}-1,3-benzothiazol-2-yl)cyclopropanecarboxamide

a) The N-(6-{[6-(tetrahydro-2H-pyran-4-ylamino)[1,2,4]triazolo[4,3-b]-pyridazin-3-yl]sulphanyl}-1,3-benzothiazol-2-yl)cyclopropanecarboxamide can be prepared in a manner similar to Example 2a, but using 300 mg of 3-[(2-amino-1,3-benzothiazol-6-yl)sulphanyl]-N-(tetrahydro-2H-pyran-4-yl)[1,2,4]-triazolo[4,3-b]pyridazin-6-amine (1a) in 3 cm³ of pyridine with 0.138 cm³ of cyclopropanecarboxylic acid chloride, after reaction for 18 h at 20° C. 292 mg of N-(6-{[6-(tetrahydro-2H-pyran-4-ylamino)[1,2,4]triazolo[4,3-b]pyridazin-3-yl]sulphanyl}-1,3-benzothiazol-2-yl)cyclopropanecarboxamide are thus obtained in the form of a beige powder, the characteristics of which are as follows:

1H NMR SPECTRUM (400 MHz, DMSO-d6) δ.ppm 0.89-1.00 (m, 4H) 1.18-1.33 (m, 2H) 1.58-1.70 (m, 2H) 1.93-2.03 (m, 1H) 3.19-3.28 (m, 2H) 3.46-3.61 (m, 1H) 3.69 (d, J=11.7 Hz, 2H) 6.79 (d, J=9.8 Hz, 1H) 7.26-7.41 (m, 2H) 7.64 (d, J=8.8 Hz, 1H) 7.95 (d, J=9.8 Hz, 1H) 8.03 (d, J=2.0 Hz, 1H) 12.66 (br, s., 1H)

MASS SPECTRUM: Waters HPLC-SQD: MH+m/z=468+; MH−=466−

### Example 4

### 1-[2-(Morpholin-4-yl)ethyl]-3-(6-{[6-(tetrahydro-2H-pyran-4-ylamino)-[1,2,4]triazolo[4,3-b]pyridazin-3-yl]sulphanyl}-1,3-benzothiazol-2-yl)urea

a) The 1-[2-(morpholin-4-yl)ethyl]-3-(6-{[6-(tetrahydro-2H-pyran-4-yl-amino)[1,2,4]triazolo[4,3-b]pyridazin-3-yl]sulphanyl}-1,3-benzothiazol-2-yl)urea can be prepared in a manner similar to Example 1a, but using 107 mg of 1-[2-(morpholin-4-yl)ethyl]-3-(6-sulphanyl-1,3-benzothiazol-2-yl)urea, 4 cm³ of degassed ethanol, 3 mg of potassium dihydrogen phosphate in 0.4 cm³ of water, 133 mg of DL-dithiothreitol and 80 mg of 3-chloro-N-(tetrahydro-2H-pyran-4-yl)[1,2,4]triazolo[4,3-b]pyridazin-6-amine (14 after 18 h at 80° C. 104 mg of 1-[2-(morpholin-4-yl)ethyl]-3-(6-{[6-(tetrahydro-2H-pyran-4-yl-amino)[1,2,4]triazolo[4,3-b]pyridazin-3-yl]sulphanyl}-1,3-benzothiazol-2-yl)urea are thus obtained in the form of a creamy-white powder, the characteristics of which are as follows:

1H NMR SPECTRUM (400 MHz, DMSO-d6) δ.ppm 1.20-1.35 (m, 2H) 1.64-1.74 (m, 2H) 2.36-2.46 (m, 6H) 3.18-3.29 (m, 4H) 3.50-3.64 (m, 5H) 3.66-3.78 (m, 2H) 6.79 (d, J=9.8 Hz, 2H) 7.29 (dd, J=8.4, 1.8 Hz, 1H) 7.37 (d, J=6.8 Hz, 1H) 7.53 (d, J=8.3 Hz, 1H) 7.94 (d, J=9.8 Hz, 1H) 7.97 (d, J=1.7 Hz, 1H) 10.87 (br. s., 1H)

MASS SPECTRUM: Waters HPLC-SQD: MH+m/z=556+; MH−=554−

b) The 1-(2-morpholin-4-ylethyl)-3-(6-sulphanyl-1,3-benzothiazol-2-yl)urea can be prepared in the following way:

a stream of argon is sparged, for 5 min, into a mixture of 900 mg of 2-{[(2-morpholin-4-ylethyl)carbamoyl]amino}-1,3-benzothiazol-6-yl thiocyanate and 40 cm³ of ethanol at 20° C. 11 mg of potassium dihydrogen phosphate in 0.4 cm³ of water and 1.1 g of DL-dithiothreitol are subsequently added. The mixture is heated at 80° C. for 3.5 h. The reaction mixture is cooled to 20° C. and then poured into water. The suspension is stirred for 45 min, with mild argon sparging being maintained. The precipitate formed is spin-filter-dried and washed with 3×10 cm³ of water, and then dried under vacuum at 20° C. 633 mg of 1-(2-morpholin-4-ylethyl)-3-(6-sulphanyl-1,3-benzothiazol-2-yl)urea are thus obtained in the form of a white solid, the characteristics of which are as follows:

MASS SPECTRUM: LC-MS-DAD-ELSD: MH+m/z=339+; (M-H)−=337−

c) The 2-{[(2-morpholin-4-ylethyl)carbamoyl]amino}-1,3-benzothiazol-6-yl thiocyanate can be prepared in the following way:

0.44 cm³ of 2-morpholin-4-ylethanamine is added, at 20° C., to a solution of 1 g of phenyl (6-thiocyanato-1,3-benzothiazol-2-yl)carbamate ester in 30 cm³ of tetrahydrofuran. After 24 h, the reaction mixture is evaporated to dryness and the residue obtained is chromatographed on a 70 g Merck cartridge (solid deposit: elution with a gradient of dichloromethane then 90/10 dichloromethane/methanol). 902 mg of 2-{[(2-morpholin-4-ylethyl)carbamoyl]amino}-1,3-benzothiazol-6-yl thiocyanate are thus recovered in the form of a colourless foam, the characteristics of which are as follows:

MASS SPECTRUM: HPLC-MS-DAD-ELSD: MH+m/z=364+

d) The phenyl (6-thiocyanato-1,3-benzothiazol-2-yl)carbamate was prepared in the following way:

7.5 g of phenyl chlorocarbonate and then 4.05 g of sodium hydrogen carbonate and 9.4 cm³ of water are added, at 20° C., to a solution of 2.5 g of commercial 2-amino-1,3-benzothiazol-6-yl thiocyanate in 94 cm³ of tetrahydrofuran. The resulting mixture is subsequently stirred at 20° C. for 20 h and then extracted with 2×150 cm³ of ethyl acetate. The organic phases are combined and then washed with 3×50 cm³ of a saturated aqueous solution of sodium hydrogen carbonate. The organic phase obtained is dried over magnesium sulphate and then concentrated to dryness under reduced pressure. The residue is taken up in 50 cm³ of water and then the resulting product is spin-filter-dried and dried under vacuum at 20° C. 3.45 g of phenyl (6-thiocyanato-1,3-benzothiazol-2-yl)carbamate are thus obtained in the form of a pale yellow solid, the characteristics of which are as follows:

MASS SPECTRUM: LC-MS-DAD-ELSD: MH+m/z=328+; (M=H)−=326−

### Example 5

### 3-Methoxy-N-(6-{[6-(tetrahydro-2H-pyran-4-ylamino)[1,2,4]triazolo-[4,3-b]pyridazin-3-yl]sulphanyl}-1,3-benzothiazol-2-yl)propanamide

a) the 3-methoxy-N-(6-{[6-(tetrahydro-2H-pyran-4-ylamino)[1,2,4]triazolo-[4,3-b]pyridazin-3-yl]sulphanyl}-1,3-benzothiazol-2-yl)propanamide can be prepared in a manner similar to Example 1a, but using 420 mg of the crude 2-[(3-methoxypropanoyl)amino]-1,3-benzothiazol-6-yl thiocyanate residue in 10 cm³ of degassed ethanol, 7 mg of potassium dihydrogen phosphate in 1 cm³ of water and 663 mg of DL-dithiothreitol, by successively heating the reaction mixture at 80° C., for 2 h, and then for 18 h after the addition of 364 mg of 3-chloro-N-(tetrahydro-2H-pyran-4-yl)[1,2,4]triazolo[4,3-b]pyridazin-6-amine (1b). 226 mg of 3-methoxy-N-(6-{[6-(tetrahydro-2H-pyran-4-ylamino)-[1,2,4]triazolo[4,3-b]pyridazin-3-yl]sulphanyl}-1,3-benzothiazol-2-yl)propanamide are thus obtained in the form of a creamy-white powder, the characteristics of which are as follows:

1H NMR MASS SPECTRUM (400 MHz, DMSO-d6) δ.ppm 1.04-1.37 (m, 2H) 1.65 (d, J=13.2 Hz, 2H) 2.73 (t, J=6.0 Hz, 2H) 3.24 (masked m, 5H) 3.42-3.61 (m, 1H) 3.60-3.76 (m, 4H) 6.79 (d, J=10.0 Hz, 1H) 7.10-7.49 (m, 2H) 7.65 (d, J=8.3 Hz, 1H) 7.95 (d, J=9.8 Hz, 1H) 8.04 (d, J=1.5 Hz, 1H) 12.39 (br. s., 1H)

MASS SPECTRUM: Waters HPLC-SQD: MH+m/z=486+; MH−=484−

b) the 2-[(3-methoxypropanoyl)amino]-1,3-benzothiazol-6-yl thiocyanate can be prepared in a manner similar to Example 2a, but using 300 mg of 2-amino-1,3-benzothiazol-6-yl thiocyanate in 9 cm³ of dichloromethane, 0.31 cm³ of triethylamine and 266 mg of 3-methoxypropanoyl chloride after 1 h at reflux. After concentration to dryness, a residue of 2-[(3-methoxypropanoyl)amino]-1,3-benzothiazol-6-yl thiocyanate is thus obtained in the form of a beige-yellow resin, which is subsequently used as it is.

### Example 6

### N²,N²-dimethyl-N-(6-{[6-(tetrahydro-2H-pyran-4-ylamino)[1,2,4]triazolo-[4,3-b]pyridazin-3-yl]sulphanyl}-1,3-benzothiazol-2-yl)glycinamide

a) the N², N²-dimethyl-N-(6-{[6-(tetrahydro-2H-pyran-4-ylamino)[1, 2, 4]triazolo-[4,3-b]pyridazin-3-yl]sulphanyl}-1,3-benzothiazol-2-yl)glycinamide can be prepared in a manner similar to Example 1a, but using 420 mg of the crude 2-[(N,N-dimethylglycyl)amino]-1,3-benzothiazol-6-yl thiocyanate residue in 10 cm³ of degassed ethanol, 7 mg of potassium dihydrogen phosphate in 1 cm³ of water and 665 mg of DL-dithiothreitol, by successively heating the reaction mixture at 80° C. for 2 h, and then for 18 h after the addition of 365 mg of 3-chloro-N-(tetrahydro-2H-pyran-4-yl)[1,2,4]triazolo[4,3-b]pyridazin-6-amine (1b). 226 mg of N²,N²-dimethyl-N-(6-{[6-(tetrahydro-2H-pyran-4-ylamino)[1,2,4]triazolo[4,3-b]pyridazin-3-yl]sulphanyl}-1,3-benzothiazol-2-yl)glycinamide are thus obtained in the form of a creamy-white powder, the characteristics of which are as follows:

1H NMR MASS SPECTRUM (400 MHz, DMSO-d6) δ.ppm 1.15-1.33 (m, 2H) 1.65 (d, J=13.0 Hz, 2H) 2.31 (s, 6H) 3.14-3.27 (masked m, 4H) 3.47-3.61 (m, 1H) 3.68 (d, J=11.2 Hz, 2H) 6.79 (d, J=10.0 Hz, 1H) 7.28-7.40 (m, 2H) 7.65 (d, J=8.6 Hz, 1H) 7.96 (d, J=9.8 Hz, 1H) 8.05 (d, J=1.2 Hz, 1H) 11.93 (br. s., 1H)

MASS SPECTRUM: Waters HPLC-SQD: MH+m/z=485+; MH−=483−

b) the 2-[(N,N-dimethylglycyl)amino]-1,3-benzothiazol-6-yl thiocyanate can be prepared in a manner similar to Example 2a, but using 300 mg of 2-amino-1,3-benzothiazol-6-yl thiocyanate in 9 cm³ of dichloromethane, 0.61 cm³ of triethylamine and 345 mg of N,N-dimethylglycyl chloride after 1 h at reflux. After concentration to dryness, a residue of 2-[(N,N-dimethylglycyl)amino]-1,3-benzothiazol-6-yl thiocyanate is thus obtained in the form of a beige yellow resin, which is subsequently used as it is.

### Example 7

### Pharmaceutical Composition

Tablets corresponding to the following formula were prepared:

| | |
|---|---|
| Product of Example 3 | 0.2 g |
| Excipient for a finished tablet weighing | 1 g |

(excipient details: lactose, talc, starch, magnesium stearate).

### Example 8

### Pharmaceutical Composition

Tablets corresponding to the following formula were prepared:

| | |
|---|---|
| Product of Example 4 | 0.2 g |
| Excipient for a finished tablet weighing | 1 g |

(excipient details: lactose, talc, starch, magnesium stearate).

Examples 3 and 4 are taken as examples of a pharmaceutical preparation, it being possible for this preparation to be carried out, if desired, with other products in the examples in the present invention.

### Pharmacological Section:

### Experimental Protocols

### I) Expression and Purification of MET, Cytoplasmic Domain

### Expression in Baculovirus:

The His-Tev-MET (956-1390) recombinant DNA in pFastBac (Invitrogen) is transfected into insect cells, and after several viral amplification steps, the final baculovirus stock is tested for the expression of the protein of interest.

After infection for 72 h at 27° C. with the recombinant virus, the SF21 cell cultures are harvested by centrifugation and the cell pellets are stored at −80° C.

### Purification:

The cell pellets are resuspended in lysis buffer (buffer A [50 mM HEPES, pH 7.5, 250 mM NaCl, 10% glycerol, 1 mM TECP]; +cocktail of protease inhibitors, Roche Diagnostics, without EDTA, ref 1873580), stirred at 4° C. until the mixture is homogeneous and then lyzed mechanically using a “Dounce” type apparatus.

After centrifugation, the lysis supernatant is incubated for 2 h at 4° C. with nickel chelate resin (His-Trap 6 Fast Flow™, GE HealthCare). After washing with 20 volumes of buffer A, the suspension is packed into a column, and the proteins are eluted with a gradient of buffer B (TpA+290 mM imidazole).

The fractions containing the protein of interest for the purpose of electrophoretic analysis (SDS PAGE) are combined, concentrated by ultrafiltration (10 kDa cut-off) and injected onto an exclusion chromatography column (Superdex™ 200, GE HealthCare) equilibrated in buffer A.

After enzymatic cleavage of the histidine tag, the protein is reinjected onto a new IMAC nickel chelate chromatography column (His-Trap 6 Fast Flow™, GE HealthCare) equilibrated in buffer A. The fractions eluted with a gradient of buffer B and containing the protein of interest after electrophoresis (SDS PAGE) are finally combined and conserved at −80° C.

For the production of autophosphorylated protein, the previous fractions are incubated for 1 h at ambient temperature after the addition of 2 mM ATP, 2 mM MgCl₂, and 4 mM Na₃VO₄. After the reaction has been stopped with 5 mM of EDTA, the reaction mixture is injected onto a HiPrep desalifying column (GE HealthCare) preequilibrated in buffer A+4 mM Na₃VO₄, and the fractions containing the protein of interest (SDS PAGE analysis) are combined and stored at −80° C. The degree of phosphorylation is verified by mass spectrometry (LC-MS) and by peptide mapping.

### II) Tests A and B

### A) Test A: HTRF MET Assay in 96-Well Format

MET at a final concentration of 5 nM is incubated in a final volume of 50 μl of enzymatic reaction in the presence of the test molecule (for a final concentration range of from 0.17 nM to 10 μM, 3% DMSO final concentration) in 10 mM MOPS buffer, pH 7.4, 1 mM DTT, 0.01% Tween 20. The reaction is initiated with the substrate solution to obtain final concentrations of 1 μg/ml poly-(GAT), 10 μM ATP and 5 mM MgCl₂. After incubation for 10 min at ambient temperature, the reaction is stopped with a 30 μl mix so as to obtain a final solution of 50 mM Hepes pH 7.5, 500 mM potassium fluoride, 0.1% BSA and 133 mM EDTA in the presence of 80 ng of streptavidin 61SAXLB Cis-Bio Int. and 18 ng of anti-phosphotyrosine Mab PT66-Europium Cryptate per well. After incubation for 2 hours at ambient temperature, the reading is taken at 2 wavelengths, 620 nm and 665 nm, on a reader for the TRACE/HTRF technique and the % inhibition is calculated from the 665/620 ratios.

The results obtained with this test A for the products of formula (I) in examples in the experimental section are such that 1050 is less than 500 nM, and in particular less than 100 nM.

B) Test B: Inhibition of the Autophosphorylation of MET; ELISA Technique (pppY1230, 1234, 1235)

a) Cell lysates: Seed MKN45 cells into 96-well plates (Cell coat BD polylysine) at 20 000 cells/well in 200 μl in RPMI medium+10% FCS+1% L-glutamine. Leave to adhere for 24 hours in an incubator.

The cells are treated the day after seeding with the products at 6 concentrations in duplicate for 1 h. At least 3 control wells are treated with the same final amount of DMSO.

Product dilution: Stock at 10 mM in pure DMSO—range from 10 mM to 30 μM with an increment of 3 in pure DMSO—intermediate 1/50 dilutions in culture medium and then removal of 10 μl added directly to the cells (200 μl): final range of 10 000 to 30 nM.

At the end of the incubation, carefully remove the supernatant and rinse with 200 μl of PBS. Next, place 100 μl of lysis buffer directly in the wells on ice and incubate at 4° C. for 30 minutes. Lysis buffer: 10 mM Tris HCl, pH7.4, 100 mM NaCl, 1 mM EDTA, 1 mM EGTA, 1% Triton X-100, 10% glycerol, 0.1% SDS, 0.5% deoxycholate, 20 mM NaF, 2 mM Na₃VO₄, 1 mM PMSF and cocktail of antiproteases.

The 100 μl of lysates are transferred into a V-bottomed polypropylene plate and the ELISA is performed immediately, or the plate is frozen at −80° C.

### b) PhosphoMET ELISA BioSource Kit KHO0281

Into each well of the kit plate, add 70 μl of kit dilution buffer+30 μL of cell lysate or 30 μl of lysis buffer for the blanks. Incubate for 2 h with gentle agitation at ambient temperature.

Rinse the wells 4 times with 400 μl of kit washing buffer. Incubate with 100 μl of anti-phospho MET antibody for 1 hour at ambient temperature.

Rinse the wells 4 times with 400 μl of kit washing buffer. Incubate with 100 μl of anti-rabbit HRP antibody for 30 minutes at ambient temperature (except for the wells of chromogen alone).

Rinse the wells 4 times with 400 μl of kit washing buffer. Introduce 100 μL of chromogen and incubate for 30 minutes in the dark at ambient temperature.

Stop the reaction with 100 μl of stop solution. Read without delay at 450 nM, 0.1 second on Wallac Victor plate reader.

### C) Test C: Measurement of Cell Proliferation by ¹⁴C-Thymidine Pulse

The cells are seeded into Cytostar 96-well plates in 180 μl for 4 hours at 37° C. and 5% CO₂: HCT116 cells at a rate of 2500 cells per well in DMEM medium+10% foetal calf serum+1% L-glutamine and MKN45 cells at a rate of 7500 cells per well in RPMI medium+10% foetal calf serum+1% L-glutamine. After these 4 hours of incubation, the products are added in 10 μl as a 20-fold concentrated solution according to the dilution method mentioned for the ELISA. The products are tested at 10 concentrations in duplicate from 10 000 nM to 0.3 nM with an increment of 3.

After treatment for 72 h, add 10 μl of ¹⁴C-thymidine at 10 μCi/ml to obtain 0.1 μCi per well. The ¹⁴C-thymidine incorporation is measured on a Micro-Beta machine (Perkin-Elmer) after 24 hours of pulse and 96 h of treatment.

All the steps of the assay are automated on BIOMEK 2000 or TECAN stations.

The results obtained with this test B for the products of formula (I) as examples in the experimental section are such that IC₅₀ is less than 10 microM and in particular less than 1 microM.

The results obtained for the products as examples in the experimental section are given in the table of pharmacological results below, as follows;

for test A, the sign + corresponds to less than 500 nM and the sign ++ corresponds to less than 100 nM;
for test B, the sign + corresponds to less than 500 nM and the sign ++ corresponds to less than 100 nM;
for test C, the sign + corresponds to less than 10 microM and the sign ++ corresponds to less than 1 microM.

### Table of Pharmacological Results:

| Example number | test A | test B | test C |
|---|---|---|---|
| 1 | ++ | | ++ |
| 2 | ++ | ++ | ++ |
| 3 | ++ | ++ | ++ |
| 4 | ++ | ++ | ++ |
| 5 | ++ | ++ | ++ |
| 6 | ++ | ++ | ++ |

1. A compound of formula (I): 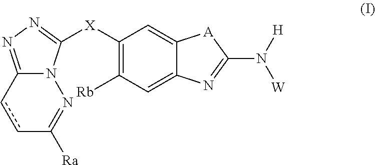
wherein [symbol]
represents a single or double bond;
Rb represents a hydrogen atom or a fluorine atom;
Ra represents an —NH-Rc radical in which Rc represents an optionally substituted heterocycloalkyl (—NH-tetrahydropyran), aryl, heteroaryl or alkylcycloalkyl radical;
X represents 5, SO or SO₂;
A represents NH or S;
W represents a hydrogen atom; an alkyl or cycloalkyl radical optionally substituted with alkoxy, heterocycloalkyl or NR3R4; or the COR radical in which R represents:
a cycloalkyl radical, or an alkyl radical, optionally substituted with an NR3R4 radical, or with an alkoxy, hydroxyl, phenyl, heteroaryl or heterocycloalkyl radical, themselves optionally substituted;
an alkoxy radical optionally substituted with NR3R4, alkoxy, hydroxyl or with heterocycloalkyl; an O-phenyl radical or an O—(CH₂)ₙ-phenyl radical, with phenyl being optionally substituted and n representing an integer from 1 to 4;
or the NR1R2 radical in which R1 and R2 are such that one of R1 and R2 represents a hydrogen atom, a cycloalkyl radical or an alkyl radical and the other of R1 and R2 represents a hydrogen atom, a cycloalkyl radical or an alkyl radical optionally substituted with one or more radicals, which may be identical or different, chosen from hydroxyl, alkoxy, heteroaryl, heterocycloalkyl, NR3R4 and optionally substituted phenyl radicals, or else R1 and R2 form, with the nitrogen atom to which they are attached, a cyclic radical containing from 3 to 10 members and optionally one or more other heteroatoms chosen from O, S, N and NH, this radical, including the possible NH that it contains, being optionally substituted;
with R3 and R4, which may be identical or different, representing a hydrogen atom or an alkyl radical, a cycloalkyl radical, a heteroaryl radical or a phenyl radical that are optionally substituted, or else R3 and R4 form, with the nitrogen atom to which they are attached, a cyclic radical containing from 3 to 10 members and optionally one or more other heteroatoms chosen from O, S, N and NH, this radical, including the possible NH that it contains, being optionally substituted;
all the cycloalkyl, heterocycloalkyl, heteroaryl, aryl and phenyl radicals defined above and also the cyclic radicals that R1 and R2 or R3 and R4 can form with the nitrogen atom to which they are attached, being optionally substituted with one or more radicals chosen from halogen atoms, hydroxyl, oxo, alkoxy, —O—CO—R5, —COOH, COOR5, —CONH₂, CONHR5, NH₂, NHR5, NR5R5′ and —NH—CO—R5 radicals and alkyl, cycloalkyl, heterocycloalkyl, CH₂-heterocycloalkyl, phenyl, CH₂-phenyl, CO-phenyl, heteroaryl and S-heteroaryl radicals, such that, in the latter radicals, the alkyl, cycloalkyl, heterocycloalkyl, phenyl and heteroaryl radicals are themselves optionally substituted with one or more radicals chosen from halogen atoms and the radicals: hydroxyl, oxo, alkyl and alkoxy containing from 1 to 4 carbon atoms, NH₂, NHalk and N(alk)₂,
all the cycloalkyl, heterocycloalkyl, heteroaryl and phenyl radicals defined above also being optionally substituted with an Si(alk)₃ radical;
R5 and R5′, which may be identical or different, represent an alkyl or cycloalkyl radical containing at most 6 carbon atoms;
alk represents an alkyl radical containing at most 4 carbon atoms;
racemic, enantiomeric and diastereoisomeric isomers, and pharmaceutically acceptable salts thereof.

2. The compound according to claim 1, wherein [symbol] represents a single or double bond;
Rb represents a hydrogen atom or a fluorine atom;
Ra represents an —NH-Rc radical in which Rc represents an optionally substituted heterocycloalkyl radical;
X represents S, SO or SO₂,
A represents NH or S;
W represents a hydrogen atom; an alkyl radical optionally substituted with alkoxy, heterocycloalkyl or NR3R4; or the COR radical in which R represents:
a cycloalkyl radical or an alkyl radical, optionally substituted with an NR3R4, alkoxy, hydroxyl, phenyl, heteroaryl or heterocycloalkyl radical, themselves optionally substituted;
an alkoxy radical optionally substituted with NR3R4, alkoxy, hydroxyl or with heterocycloalkyl; an O-phenyl radical or an O—(CH₂)ₙ-phenyl radical, with phenyl being optionally substituted and n representing an integer from 1 to 4;
or the NR1R2 radical in which R1 and R2 are such that one of R1 and R2 represents a hydrogen atom, a cycloalkyl radical or an alkyl radical and the other of R1 and R2 represents a hydrogen atom, a cycloalkyl radical or an alkyl radical optionally substituted with one or more radicals, which may be identical or different, chosen from hydroxyl, alkoxy, heteroaryl, heterocycloalkyl, NR3R4 and optionally substituted phenyl radicals, or else R1 and R2 form, with the nitrogen atom to which they are attached, a cyclic radical containing from 3 to 10 members and optionally one or more other heteroatoms chosen from O, S, N and NH, this radical, including the possible NH that it contains, being optionally substituted;
with R3 and R4, which may be identical or different, representing a hydrogen atom or an alkyl radical, a cycloalkyl radical, a heteroaryl radical or a phenyl radical that is optionally substituted, or else R3 and R4 form, with the nitrogen atom to which they are attached, a cyclic radical containing from 3 to 10 members and optionally one or more other heteroatoms chosen from O, S, N and NH, this radical, including the possible NH that it contains, being optionally substituted;
all the cycloalkyl, heterocycloalkyl, heteroaryl, aryl and phenyl radicals defined above and also the cyclic radicals that R1 and R2 or R3 and R4 can form with the nitrogen atom to which they are attached, being optionally substituted with one or more radicals chosen from halogen atoms, hydroxyl, oxo, alkoxy, —O—CO—R5, NH₂, NHalk and N(alk)₂ radicals and alkyl, cycloalkyl, heterocycloalkyl, CH₂-heterocycloalkyl, phenyl, CH₂-phenyl, CO-phenyl, heteroaryl and S-heteroaryl radicals, such that, in the latter radicals, the alkyl, cycloalkyl, heterocycloalkyl, phenyl and heteroaryl radicals are themselves optionally substituted with one or more radicals chosen from halogen atoms and the radicals: hydroxyl, oxo, alkyl and alkoxy containing from 1 to 4 carbon atoms, NH₂, NHalk and N(alk)₂,
R5 represents an alkyl or cycloalkyl radical containing at most 6 carbon atoms;
racemic, enantiomeric and diastereoisomeric isomers, and pharmaceutically acceptable salts thereof.

3. The compound according to claim 1, wherein
A represents NH or S;
W represents a hydrogen atom; an alkyl radical optionally substituted with alkoxy, heterocycloalkyl or NR3R4; or the COR radical in which R represents:
a cycloalkyl radical or an alkyl radical optionally substituted with an NR3R4, alkoxy, hydroxyl, phenyl or heterocycloalkyl radical, themselves optionally substituted;
an alkoxy radical optionally substituted with NR3R4, alkoxy, hydroxyl or with heterocycloalkyl; an O-phenyl radical or an O—(CH₂)ₙ-phenyl radical, with phenyl being optionally substituted and n representing an integer from 1 to 4;
or the NR1R2 radical, in which R1 and R2 are such that one of R1 and R2 represents a hydrogen atom or an alkyl radical and the other of R1 and R2 represents a hydrogen atom, a cycloalkyl radical or an alkyl radical optionally substituted with an alkoxy, heterocycloalkyl or NR3R4 radical; or else R1 and R2 form, with the nitrogen atom to which they are attached, a cyclic radical containing from 3 to 10 members and optionally one or more other heteroatoms chosen from O, S, N and NH, this radical, including the possible NH that it contains, being optionally substituted;
with NR3R4 such that R3 and R4, which may be identical or different, represent a hydrogen atom or an alkyl radical, or else R3 and R4 form, with the nitrogen atom to which they are attached, a cyclic radical containing from 3 to 10 members and optionally one or more other heteroatoms chosen from O, S, N and NH, this radical, including the possible NH that it contains, being optionally substituted;
all the cycloalkyl, heterocycloalkyl and phenyl radicals and also the cyclic radicals that R1 and R2 or R3 and R4 can form with the nitrogen atom to which they are attached, defined above, being optionally substituted with one or more radicals chosen from halogen atoms, hydroxyl, alkoxy, NH₂, NHalk and N(alk)₂ radicals and alkyl, heterocycloalkyl, CH₂-heterocycloalkyl, phenyl, CH₂-phenyl and heteroaryl radicals, such that, in the latter radicals, the alkyl, heterocycloalkyl, phenyl and heteroaryl radicals are themselves optionally substituted with one or more radicals chosen from halogen atoms and the radicals: hydroxyl, alkyl and alkoxy containing from 1 to 4 carbon atoms, NH₂, NHalk and N(alk)₂;
racemic, enantiomeric and diastereoisomeric isomers, and pharmaceutically acceptable salts thereof.

4. The compound according to claim 1, wherein
A represents NH or S;
W represents a hydrogen atom; an alkyl radical optionally substituted with a heterocycloalkyl or NR3R4 radical; or the COR radical in which R represents:
a cycloalkyl radical or an alkyl radical which are optionally substituted with an NR3R4 or alkoxy radical;
an O-phenyl radical or an O—(CH₂)ₙ-phenyl radical, with phenyl optionally substituted and n representing an integer from 1 to 2;
or the NR1R2 radical, in which R1 and R2 are such that one of R1 and R2 represents a hydrogen atom, a cycloalkyl radical or an alkyl radical and the other of R1 and R2 represents a hydrogen atom, an alkyl radical optionally substituted with a heterocyclic or NR3R4 radical, or else R1 and R2 form with the nitrogen atom to which they are attached, a cyclic radical optionally containing one or more other heteroatoms chosen from O, S, N and NH, this radical, including the possible NH that it contains, being optionally substituted;
with NR3R4 such that R3 and R4, which may be identical or different, represent a hydrogen atom or an alkyl radical, or else R3 and R4 form, with the nitrogen atom to which they are attached, a cyclic radical optionally containing one or more other heteroatoms chosen from O, S, N and NH, this radical, including the possible NH that it contains, being optionally substituted;
all the cycloalkyl, heterocyclic and phenyl radicals and also the cyclic radicals that R1 and R2 or R3 and R4 can form with the nitrogen atom to which they are attached, defined above, being optionally substituted with one or more radicals chosen from halogen atoms, hydroxyl, alkoxy, NH₂, NHalk and N(alk)₂ radicals and alkyl and phenyl radicals, the latter radicals themselves being optionally substituted with one or more radicals chosen from halogen atoms and the radicals: hydroxyl, alkyl and alkoxy containing from 1 to 4 carbon atoms, NH₂, NHalk and N(alk)₂;
racemic, enantiomeric and diastereoisomeric isomers, and pharmaceutically acceptable salts thereof.

5. The compound according to claim 1, wherein A represents NH, racemic, enantiomeric and diastereoisomeric isomers, and pharmaceutically acceptable salts thereof.

6. The compound according to claim 1, wherein A represents S, racemic, enantiomeric and diastereoisomeric isomers, and pharmaceutically acceptable salts thereof.

7. The compound according to claim 1, corresponding to formula (Ia) or (Ib): 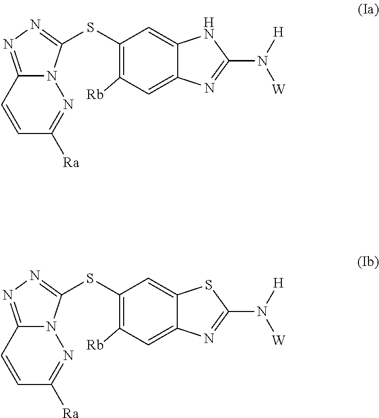
racemic, enantiomeric and diastereoisomeric isomers, and pharmaceutically acceptable salts thereof.

8. The compound according to claim 1, wherein [symbol] represents a double bond, corresponding to formula (I″): 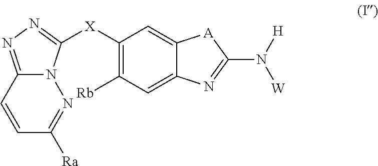
racemic, enantiomeric and diastereoisomeric isomers, and pharmaceutically acceptable salts thereof.

9. The compound according to claim 1, wherein [symbol] represents a double bond, corresponding to formula (I″a): 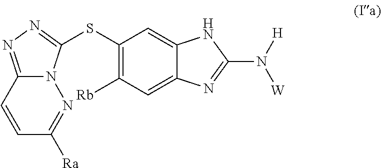
racemic, enantiomeric and diastereoisomeric isomers, and pharmaceutically acceptable salts thereof.

10. The compound according to claim 1, wherein [symbol] represents a double bond, corresponding to formula (I″b): 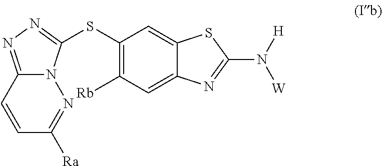
racemic, enantiomeric and diastereoisomeric isomers, and pharmaceutically acceptable salts thereof.

11. The compound according to claim 1, wherein [symbol]
represents a single or double bond;
Ra represents an optionally substituted —NH-heterocycloalkyl radical;
Rb represents a hydrogen atom;
X represents S;
A represents S;
W represents a hydrogen atom; or the COR radical in which R represents:
a cycloalkyl radical, or an alkyl radical, optionally substituted with an NR3R4 radical, or with an alkoxy radical;
or the NR1R2 radical in which R1 and R2 are such that one represents a hydrogen atom and the other of R1 and R2 represents an alkyl radical optionally substituted with a heterocycloalkyl radical;
with NR3R4 such that R3 and R4, which may be identical or different, represent a hydrogen atom or an alkyl radical;
all the heterocycloalkyl radicals defined above being optionally substituted with one or more radicals chosen from halogen atoms, hydroxyl, oxo, alkoxy, NH₂, NHalk and N(alk)₂ radicals and alkyl and phenyl radicals which are themselves optionally substituted with one or more radicals chosen from halogen atoms and the radicals: hydroxyl, oxo, alkyl and alkoxy containing from 1 to 4 carbon atoms, NH₂, NHalk and N(alk)₂;
racemic, enantiomeric and diastereoisomeric isomers, and pharmaceutically acceptable salts thereof.

12. The compound according to claim 1, corresponding to the following formulae:
3-[(2-amino-1,3-benzothiazol-6-yl)sulphanyl]-N-(tetrahydro-2H-pyran-4-yl)[1,2,4]triazolo[4,3-b]pyridazin-6-amine
N-(6-{[6-(tetrahydro-2H-pyran-4-ylamino) [1,2,4]triazolo[4,3-b]pyridazin-3-yl]sulphanyl}-1,3-benzothiazol-2-yl)acetamide
N-(6-{[6-(tetrahydro-2,4-pyran-4-ylamino)[1,2,4]triazolo[4,3-b]pyridazin-3-yl]sulphanyl}-1,3-benzothiazol-2-yl)cyclopropanecarboxamide
1-[2-(morpholin-4-yl)ethyl]-3-(6-{[6-(tetrahydro-2H-pyran-4-ylamino)[1,2,4]triazolo[4,3-b]pyridazin-3-yl]sulphanyl}-1,3-benzothiazol-2-yl)urea
3-methoxy-N-(6-{[6-(tetrahydro-2H-pyran-4-ylamino)[1,2,4]triazolo[4,3-b]pyridazin-3-yl]sulphanyl}-1,3-benzothiazol-2-yl)propanamide
N²,N²-dimethyl-N-(6-{[6-(tetrahydro-2H-pyran-4-ylamino)[1,2,4]triazolo[4,3-b]pyridazin-3-yl]sulphanyl}-1,3-benzothiazol-2-yl)glycinamide
and pharmaceutically acceptable salts thereof.

13. A process for preparing the compound according to claim 1.

14. A process for preparing the compound according to claim 1, wherein A represents NH.

15. A process for preparing the compound according to claim 1, wherein A represents S.

16. A pharmaceutical composition comprising the compound of claim 1, and pharmaceutically acceptable salts thereof.

17. A pharmaceutical composition comprising the compound of claim 12, and pharmaceutically acceptable salts thereof.

18. A pharmaceutical composition containing, as active ingredient, at least one compound according to claim 1, or a pharmaceutically acceptable salt of said compound or a prodrug of said compound, and a pharmaceutically acceptable carrier.

19. A method of inhibiting the activity of the MET protein kinase and mutant forms thereof in a patient in need thereof comprising administering to said patient a therapeutically effective amount of the compound according to claim 1.

20. The method according to claim 19, wherein the protein kinase is in a cell culture.

21. The method according to claim 19, wherein the protein kinase is in a mammal.

22. A method of treating or preventing a disease chosen from the following group: blood vessel proliferation disorders, fibrotic disorders, ‘mesangial’ cell proliferation disorders, metabolic disorders, allergies, asthma, thrombosis, nervous system diseases, retinopathy, psoriasis, rheumatoid arthritis, diabetes, muscle degeneration and cancers in a patient in need thereof comprising administering to said patient a therapeutically effective amount of the compound according to claim 1.

23. A method of treating cancers in a patient in need thereof comprising administering to said patient a therapeutically effective amount of the compound according to claim 1.

24. The method according to claim 23, wherein solid or liquid tumours are treated.

25. The method according to claim 23, wherein said cancers are resistant to cytotoxic agents.

26. The method according to claim 23, comprising treating of primary tumours and/or metastases, in particular in gastric, hepatic, renal, ovarian, colon, prostate and lung (NSCLC and SCLC) cancers, glioblastomas, thyroid, bladder or breast cancers, in melanomas, in lympyhoic or myeloid hematopoietic tumours, in sarcomas, in brain, larynx or lymphatic system cancers, bone cancers and pancreatic cancers.

27. (canceled)

28. (canceled)

29. The compound according to claim 1, wherein said compound is a kinase inhibitor.

30. The compound according to claim 1, wherein said compound is a MET inhibitor.

31. A product having one of the following formulas: 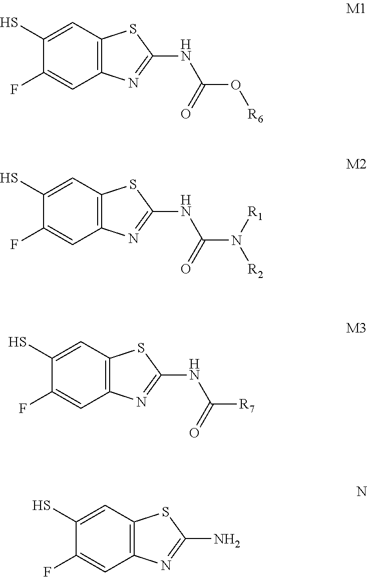
in which the CONR1R2, CO₂R6 and COR7 groups, which constitute W, can take the values of W as defined above for the products of formula (I) in claim 1, when W≠H.
